(19) United States
(12) Patent Application Publication
(10) Pub. No.: US 2013/0274648 A1
(43) Pub. Date: Oct. 17, 2013

(54) BLOOD FLOW CONTROLLERS AND METHODS

(71) Applicant: Judah Weinberger, Teaneck, NJ (US)

(72) Inventor: Judah Weinberger, Teaneck, NJ (US)

(73) Assignee: The Trustees of Columbia University in the City of New York, New York, NY (US)

(21) Appl. No.: 13/812,380

(22) PCT Filed: Jul. 25, 2011
(86) PCT No.: PCT/US11/45216

§ 371 Date: Jul. 3, 2013

(30) Foreign Application Priority Data
Jul. 26, 2010 (US) 61367755

(51) Int. Cl. A61M 27/00

(52) U.S. Cl. CPC A61M 27/002
USPC 604/9

(57) ABSTRACT

Blood flow restrictors are discussed. In some examples, a blood flow restrictor apparatus is configured to be placed within a destination vessel for an arteriovenous hemodialysis access. In an example, the apparatus includes a tubular portion including a delivery configuration and a deployed configuration. In a further example, a size-limiting portion is configured to constrain a size of the tubular portion in the deployed configuration. The tubular portion, in some examples, includes a first portion including a first size substantially matching an interior size of the destination vessel with the tubular portion in the deployed configuration. In another example, the tubular portion includes a second portion constrained by the size-limiting portion to include a second size smaller than the first size of the tubular portion.

### CLAIM OF PRIORITY

This patent application claims the benefit of priority of U.S. Provisional Patent Application Ser. No. 61/367,755, entitled BLOOD FLOW CONTROLLERS AND METHODS, filed on Jul. 26, 2010 (Attorney Docket No. 2413.120PRV), which is incorporated by reference herein in its entirety.

### TECHNICAL FIELD

This patent document pertains generally to vascular access systems, apparatuses, and methods. More particularly, but not by way of limitation, this patent document pertains to arteriovenous hemodialysis access blood flow controllers and methods.

### BACKGROUND

A number of medical procedures, such as hemodialysis, chemotherapy, transfusions, etc., require repeated access to a subject's vascular anatomy. In hemodialysis, for example, blood is removed from the subject's artery, treated with a dialysis machine that cleanses the blood of toxins (such as potassium and urea, as well as free water), and introduced back into the subject at a vein. Hemodialysis is typically conducted in a dedicated facility, either in a special room in a hospital or a clinic that specializes in hemodialysis. Hemodialysis sessions typically last about 3-6 hours and occur about 3 times per week for the duration of the subject's life or until the subject receives a kidney transplant.

For hemodialysis to be effective, large volumes of blood must be removed rapidly from the subject's body, passed through the dialysis machine, and returned to the subject. A number of operations have been developed to provide access to the circulatory system of a subject to connect the subject to the dialysis machine. The three primary modes of access to the blood in hemodialysis include an intravenous catheter, an arteriovenous fistula, or an arteriovenous graft. The type of access is typically influenced by factors such as the degree of the subject's renal (i.e., kidney) failure or the condition of his or her vasculature.

Catheter access typically consists of a plastic catheter with two lumens. The catheter is inserted into a large vein (typically in a limb) to allow withdrawal of relatively large flows of blood using one lumen. This blood is fed through the dialysis device, and returned to the subject via the other lumen. However, using the catheter access mode almost always allows less blood flow than that of a well functioning arteriovenous fistula or graft.

Arteriovenous fistulas and grafts comprise second and third modes, respectively, of access to blood in hemodialysis. To create an arteriovenous fistula, a vascular surgeon joins an artery and a vein together (typically in an upper extremity) through anastomosis. Since this bypasses the capillaries, blood flows at a very high rate through the arteriovenous fistula as compared to typical vessel flow. During treatment, two needles or cannulas are inserted into the arteriovenous fistula, one to draw blood and the other to return it. The advantages of arteriovenous fistula use include relative absence of a potential foreign body reaction, as there is no exogenous material involved in their formation, and higher blood flow rates that translate to more effective dialysis. However, if an arteriovenous fistula permits very high flow, then excessive “blood steal” can result in inadequate flow to the distal extremities of that limb. This may result in cold extremities of such limb, cramping pains, or tissue damage.

Arteriovenous grafts are much like arteriovenous fistulas, except that an artificial vessel made of a synthetic material is used to join the artery and vein. As such, arteriovenous grafts may result in foreign body reactions. However, arteriovenous grafts can typically be ready for use as a dialysis conduit soon after surgical implantation, unlike arteriovenous fistulas. Arteriovenous grafts are often used when the subject's native vasculature does not permit using an arteriovenous fistula.

### OVERVIEW

While the high blood flow rates of arteriovenous fistulas and grafts are thought to reduce the likelihood of thrombosis, there can be a number of complications including high output heart failure and a distal blood steal syndrome resulting from such flow. In addition, very high flow may result in thrombosis resulting from venous hyperplasia or stenosis occurring either at the graft-vein anastomosis or centrally in the subclavian or axillary veins.

In Example 1, a blood flow restrictor apparatus is configured to be placed within a destination vessel for an arteriovenous hemodialysis access. The apparatus comprises a tubular portion including a delivery configuration and a deployed configuration. The tubular portion is deformably expandable from the delivery configuration to the deployed configuration. A size-limiting portion is configured to constrain a size of the tubular portion when expanded in the deployed configuration. The tubular portion includes a first portion including a first size substantially matching an interior size of the destination vessel with the tubular portion expanded in the deployed configuration. The tubular portion, when expanded in the deployed configuration, includes a second portion constrained by the size-limiting portion to include a second size smaller than the first size of the tubular portion.

In Example 2, the blood flow restrictor apparatus of Example 1 is optionally configured such that the tubular portion includes a first end and a second end. The first portion of the tubular portion includes at least one of the first and second ends of the tubular portion.

In Example 3, the blood flow restrictor apparatus of at least one of Example 1 or 2 is optionally configured such that the tubular portion includes a stent graft.

In Example 4, the blood flow restrictor apparatus of Example 3 is optionally configured such that the size-limiting portion includes a stent disposed around the tubular portion proximate the second portion.

In Example 5, the blood flow restrictor apparatus of Example 3 is optionally configured such that the size-limiting portion includes a fixed ring disposed around the tubular portion proximate the second portion.

In Example 6, the blood flow restrictor apparatus of at least one of Examples 1-5 is optionally configured such that the size-limiting portion includes a stent.

In Example 7, the blood flow restrictor apparatus of Example 6 is optionally configured such that the stent includes a first weave pattern and a second weave pattern. The first weave pattern is configured to constrain the first portion of the tubular portion to the first size, and the second weave pattern is configured to constrain the second portion of the tubular portion to the second size.

In Example 8, the blood flow restrictor apparatus of Example 6, is optionally configured such that the stent includes a first molded portion and a second molded portion. The first molded portion includes a different expansion characteristic from the second molded portion. The first molded portion is configured to constrain the first portion of the tubular portion to the first size, and the second molded portion is configured to constrain the second portion of the tubular portion to the second size.

In Example 9, the blood flow restrictor apparatus of at least one of Examples 1-8 is optionally configured such that the first portion of the tubular portion includes the first and second ends, and the second portion of the tubular portion is disposed between the first and second ends.

In Example 10, the blood flow restrictor apparatus of at least one of Examples 1-9 is optionally configured such that the destination vessel includes an arteriovenous graft.

In Example 11, the blood flow restrictor apparatus of at least one of Examples 1-10 is optionally configured such that the destination vessel includes a vessel of an arteriovenous fistula.

In Example 12, the blood flow restrictor apparatus of Example 11 is optionally configured such that the destination vessel includes a vein of the arteriovenous fistula.

In Example 13, the blood flow restrictor apparatus of at least one of Examples 1-12 is optionally configured such that the tubular portion and the size-limiting portion are integrally formed together.

In Example 14, the blood flow restrictor apparatus of at least one of Examples 1-13 is optionally configured such that the size-limiting portion is frictionally attached to the tubular portion.

In Example 15, the blood flow restrictor apparatus of at least one of Examples 1-14 is optionally configured such that the size-limiting portion is magnetically attached to the tubular portion.

In Example 16, the blood flow restrictor apparatus of at least one of Examples 1-15 is optionally configured such that the tubular portion includes a pin extending from a surface of the tubular portion. The pin is configured to maintain the size-limiting portion in place on the tubular portion.

In Example 17, the blood flow restrictor apparatus of Example 16 is optionally configured such that the tubular portion includes two pins configured to retain the size-limiting portion in place on the tubular portion between the two pins.

In Example 18, the blood flow restrictor apparatus of at least one of Examples 1-17 is optionally configured such that the tubular portion in the deployed configuration includes: an entry portion converging from the first size at a first end to the second size of the tubular portion, and an exit portion diverging from the second size to the first size at a second end of the tubular portion.

In Example 19, the blood flow restrictor apparatus of at least one of Examples 1-18 optionally comprises an imageable feature configured to be viewable in an imaging modality.

In Example 20, the blood flow restrictor apparatus of Example 19 is optionally configured such that the imageable feature includes a radio opaque feature.

In Example 21, the blood flow restrictor apparatus of Example 19 is optionally configured such that the imageable feature includes a ultrasound reflective feature.

In Example 22, the blood flow restrictor apparatus of at least one of Examples 1-21 is optionally configured such that the tubular portion is configured to self-expand from the delivery configuration to the deployed configuration.

In Example 23, the blood flow restrictor apparatus of at least one of Examples 1-22 is optionally configured such that the tubular portion is configured to fit over a balloon configured to expand the tubular portion from the delivery configuration to the deployed configuration.

In Example 24, the blood flow restrictor apparatus of Example 23 is optionally configured such that the balloon includes two balloon portions. A first balloon portion is configured to expand the tubular portion proximate a first end. A second balloon portion is configured to expand the tubular portion proximate a second end.

In Example 25, the blood flow restrictor apparatus of at least one of Examples 1-24 is optionally configured such that the blood flow restrictor apparatus with the tubular portion in the delivery configuration is configured to fit within a dialysis introducer.

In Example 26, a method of restricting blood flow for an arteriovenous hemodialysis access comprises placing a blood flow restrictor apparatus, in a delivery configuration, within a destination vessel for the arteriovenous hemodialysis access. The blood flow restrictor apparatus is expanded to a deployed configuration, wherein the blood flow restrictor apparatus includes a tubular portion and a size-limiting portion. The size-limiting portion is configured to constrain a size of the tubular portion when expanded in the deployed configuration. The tubular portion includes a first portion including a first size substantially matching an interior size of the destination vessel with the tubular portion expanded in the deployed configuration. The tubular portion, when expanded in the deployed configuration, includes a second portion constrained by the size-limiting portion to include a second size smaller than the first size of the tubular portion.

In Example 27, the method of Example 26 is optionally configured such that expanding the blood flow restrictor apparatus to a deployed configuration includes inflating a balloon to expand the blood flow restrictor apparatus.

In Example 28, the method of Example 27 is optionally configured such that inflating the balloon includes inflating two balloon portions, wherein a first balloon portion is configured to expand the tubular portion proximate a first end, and a second balloon portion is configured to expand the tubular portion proximate a second end.

In Example 29, the method of at least one of Examples 26-28 is optionally configured such that expanding the blood flow restrictor apparatus to a deployed configuration includes allowing the blood flow restrictor apparatus to self-expand.

In Example 30, the method of at least one of Examples 26-29 is optionally configured such that placing the blood flow restrictor apparatus within the destination vessel includes using a catheter to place the blood flow restrictor apparatus within the destination vessel.

In Example 31, the method of at least one of Examples 26-30 is optionally configured such that placing the blood flow restrictor apparatus within the destination vessel includes using a vascular sheath to place the blood flow restrictor apparatus within the destination vessel.

In Example 32, the method of at least one of Examples 26-31 is optionally configured such that placing the blood flow restrictor apparatus within the destination vessel includes placing the blood flow restrictor apparatus within an arteriovenous graft.

In Example 33, the method of at least one of Examples 26-32 is optionally configured such that placing the blood flow restrictor apparatus within the destination vessel includes placing the blood flow restrictor apparatus within a vessel of an arteriovenous fistula.

In Example 34, the method of at least one of Examples 26-33 is optionally configured such that placing the blood flow restrictor apparatus within the destination vessel includes placing the blood flow restrictor apparatus within a vein of the arteriovenous fistula.

### BRIEF DESCRIPTION OF THE DRAWINGS

In the drawings, which are not necessarily drawn to scale, like numerals describe similar components throughout the several views. The drawings illustrate generally, by way of example, but not by way of limitation, various embodiments discussed in the present patent document.

FIG. 1 is a plan view of a hemodialysis system and an environment in which the hemodialysis system can generally be used.

FIG. 2A is a schematic view of an arteriovenous graft and an environment in which the graft can be used, as constructed in accordance with an embodiment.

FIG. 2B is a schematic view of an arteriovenous graft system and an environment in which the graft system can be used, as constructed in accordance with an embodiment.

FIG. 2C is a detailed view of an arteriovenous graft system and an environment in which the graft system can be used, as constructed in accordance with an embodiment.

FIG. 3A is a schematic view of portions of an arteriovenous graft system, as constructed in accordance with an embodiment.

FIG. 3B is a side cross-sectional view along line 3B-3B of FIG. 3A illustrating interior portions of the arteriovenous graft system of FIG. 3A.

FIG. 3C is a transverse cross-sectional view along line 3C-3C of FIG. 3A illustrating the varying diameters of the arteriovenous graft system of FIG. 3A.

FIG. 4A is a schematic view of portions of an arteriovenous graft system, as constructed in accordance with an embodiment.

FIG. 4B is a side cross-sectional view along line 4B-4B of FIG. 4A illustrating interior portions of the arteriovenous graft system of FIG. 4A.

FIG. 4C is a transverse cross-sectional view along line 4C-4C of FIG. 4A illustrating the varying diameters of the arteriovenous graft system of FIG. 4A.

FIGS. 5A-5C illustrate insertion of an example restrictor apparatus into an arteriovenous hemodialysis access, in accordance with an embodiment.

FIGS. 6A-6B illustrate an example restrictor apparatus, as constructed in accordance with an embodiment.

FIGS. 7A-7C illustrate insertion of an example restrictor apparatus into an arteriovenous hemodialysis access, in accordance with an embodiment.

FIGS. 8A-8D illustrate insertion of an example restrictor apparatus into an arteriovenous hemodialysis access, in accordance with an embodiment.

FIGS. 9A-9D illustrate insertion of an example restrictor apparatus into an arteriovenous hemodialysis access, in accordance with an embodiment.

FIG. 10 is a summary chart from a computer simulation listing blood flow properties when using and not using a restrictor apparatus, as constructed in accordance with an embodiment.

FIG. 11A is a schematic view of a hemodialysis system not including a restrictor apparatus and one or more measurement devices used for in vivo experimentation, as constructed in accordance with an embodiment.

FIG. 11B is a schematic view of a hemodialysis system including a restrictor apparatus and one or more measurement devices used for in vivo experimentation, as constructed in accordance with an embodiment.

FIGS. 11C-11E provide a data chart summarizing in vivo experimentation results of a hemodialysis system including and not including a restrictor apparatus, as constructed in accordance with an embodiment.

FIG. 12 illustrates an example method of forming an arteriovenous graft system, including forming a restrictor apparatus having fixed dimensions.

FIG. 13 illustrates an example method of restricting a flow of blood through an arteriovenous graft system.

FIGS. 14A-14B illustrate an example restrictor apparatus for an arteriovenous hemodialysis access, in accordance with an embodiment.

FIGS. 15A-15B illustrate an example restrictor apparatus for an arteriovenous hemodialysis access, in accordance with an embodiment.

FIGS. 16A-16J illustrate example restrictor apparatus components for an arteriovenous hemodialysis access, in accordance with various embodiments.

FIGS. 17A-17D illustrate insertion of an example restrictor apparatus into an arteriovenous hemodialysis access, in accordance with an embodiment.

FIGS. 18A-18D illustrate insertion of an example restrictor apparatus into an arteriovenous hemodialysis access, in accordance with an embodiment.

### DETAILED DESCRIPTION

Healthy kidneys not only clean blood by filtering out extra water and wastes, but they also produce hormones that help maintain strong bones and healthy blood. When a subject's kidneys fail, numerous debilitating effects are experienced by the subject, including rising blood pressure, accumulation of fluids and toxic wastes in the subject's body and insufficient red blood cell production. Treatment is therefore required to artificially replace the work of the failed kidneys.

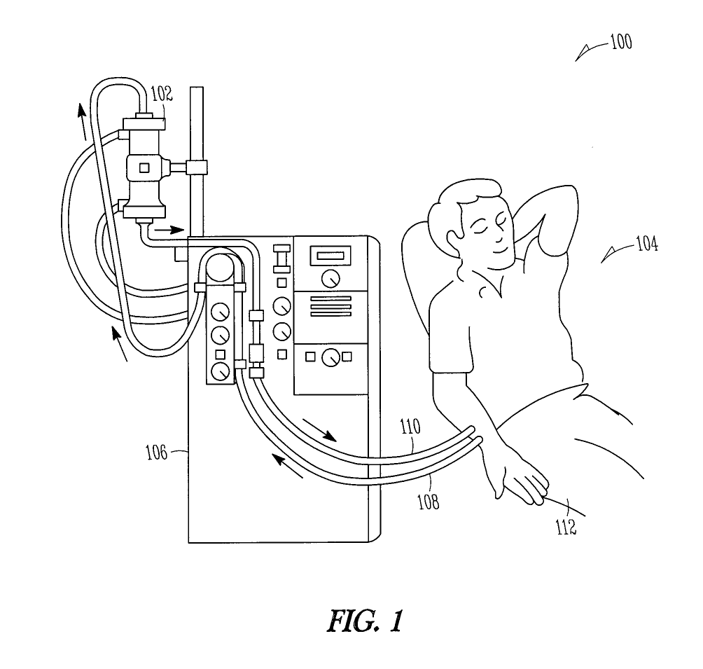

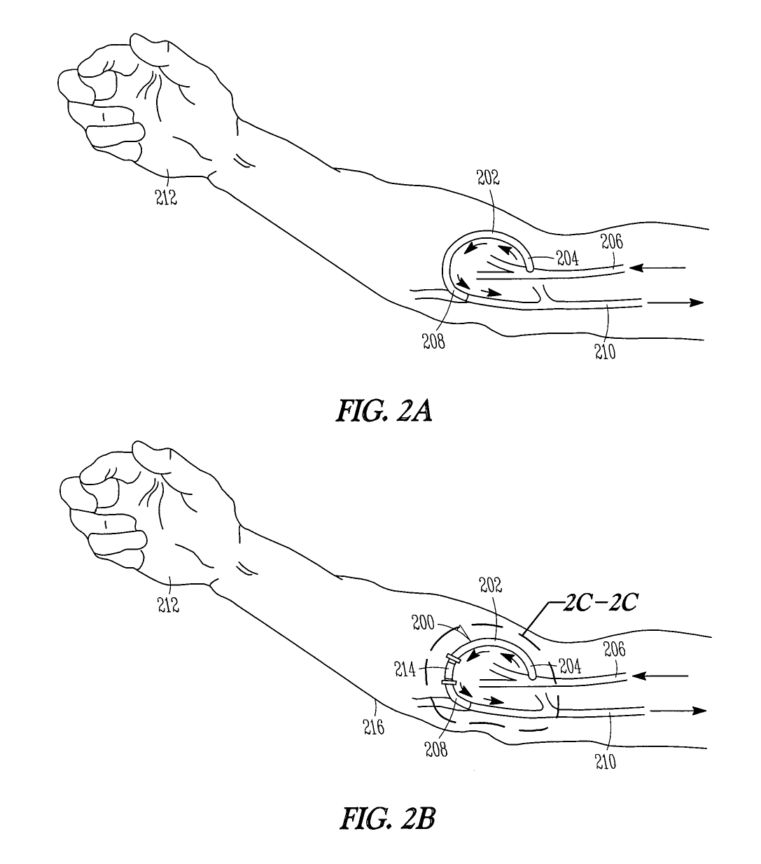

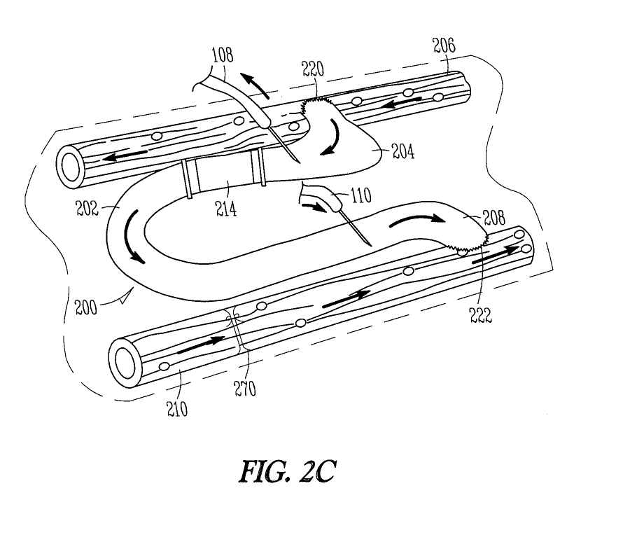

A hemodialysis machine acts as an artificial kidney to remove toxins and water from the subject's blood. Hemodialysis generally uses a special filter, typically a dialyzer 102, to clean the blood. FIG. 1 illustrates a hemodialysis system 100 and a subject 104 with which the hemodialysis system 100 can be used. The hemodialysis system 100, in this example, generally includes a dialysis machine 106, one or more cannulas 108, 110, and an arteriovenous hemodialysis access, such as an arteriovenous graft 202 (FIG. 2A). As shown in FIG. 2A, the arteriovenous graft 202 extends from an arterial end portion 204, which can be anastomosed with a subject's artery 206, to a venous end portion 208, which can be anastomosed with a subject's vein 210.

As shown in FIG. 2C, an arterial cannula 108 and a venous cannula 110 can be inserted into the arteriovenous graft 202 near the graft-artery anastomosis 220 and graft-vein anastomosis 222, respectively. Then, as shown in FIG. 1, blood from the subject 104 can be drawn via the arterial cannula 108 at the arterial side of the arteriovenous graft and received by the dialysis machine 106 where it is dialyzed (i.e., cleansed). After being dialyzed, the blood can be returned to the subject 104 at the venous side of the arteriovenous graft via the venous cannula 110.

To filter the blood efficiently, the dialysis machine 106 typically requires a blood flow rate of about 400 cubic centimeters per minute (i.e., 400 cc/min). To supply such a high blood flow rate while preventing vessel wall collapse as the dialysis machine 106 extracts blood, a relatively large diameter graft (e.g., a graft about 6 millimeters in tubular interior diameter) is used. However, such large diameter grafts can cause high output heart failure, atrophy of one or more peripheral limbs, such as a hand 212 (FIG. 2A), or thrombosis secondary to venous hyperplasia or stenosis occurring either at the graft-vein anastomosis 222 (FIG. 2C) or centrally in the subclavian or axillary veins.

The present inventors have recognized a need for, among other things, cost-effective vascular access systems, apparatuses, and methods that reduce the excess circulatory load obligated by a relatively large diameter arteriovenous graft 202 and lessen the blood steal of such graft 202 by reducing flow through it, without encouraging clotting, and while still maintaining a high flow rate during dialysis. Accordingly, the present inventors have developed a blood flow restrictor apparatus 214 for use with the arteriovenous graft 202 (collectively referred to as an arteriovenous graft system 200 (see, e.g., FIGS. 2B, 2C, 3A, 3B, 4A, and 4B)). The restrictor apparatus 214 is sized and shaped to, among other things, reduce the basal (non-hemodialysis state) blood flow through the arteriovenous graft 202, while still allowing the flow rates typical for efficient dialysis. In some examples, the restrictor apparatus 214 can, additionally or alternatively, reduce recirculation of dialyzed blood, thereby facilitating the obtaining of cleaner blood in less time. Reducing recirculation of dialyzed blood increases hemodialysis efficiency, which can lessen the hemodialysis treatment time requirements for subjects 104 with renal failure.

### EXAMPLES

An example of a right arm 216 of a subject 104 (FIG. 1) subcutaneously implanted with an arteriovenous graft system 200 is shown in FIG. 2B. In this example, the arteriovenous graft system 200 includes a tubular or similar arteriovenous graft 202 and an integral or separable blood flow restrictor apparatus 214. The arteriovenous graft system 200 is generally connected between a subject's artery 206, such as one of the brachial, ulnar, or radial arteries, and a subject's vein 210, such as the cephalic vein.

FIG. 2C illustrates in more detail, a portion of the subject's right arm 216 and the arteriovenous graft system 200 subcutaneously implanted therein. The arteriovenous graft system 200 provides a shunted path of low blood flow resistance that allows a substantial portion of the arterial blood flowing through the subject's artery 206 to be diverted at the graft-artery sewn anastomosis 220, through the arteriovenous graft 202 and restrictor apparatus 214, to the subject's vein 210 at the graft-vein sewn anastomosis 222, such as during the blood diversion of a hemodialysis session.

During hemodialysis, an arterial cannula 108 and a venous cannula 110 are inserted into the arteriovenous graft 202 near the graft-artery anastomosis 220 and the graft-vein anastomosis 222, respectively. Blood is drawn from the subject 104 (FIG. 1) upstream of the restrictor apparatus 214 via the arterial cannula 108 at the arterial end portion 204 of the arteriovenous graft 202, sent through a dialysis machine 106 (FIG. 1) where it is dialyzed, and returned to the subject 104 downstream of the restrictor apparatus 214 at the venous end portion 208 of the arteriovenous graft 202 via the venous cannula 110. In one example, but as may vary, the venous cannula 110 and the arterial cannula 108 are inserted into the subject's skin about 2-3 centimeters or more apart, which translates to about 8-10 centimeters or more separation on the arteriovenous graft 202 due to a U-shape implantation configured, such as is shown in FIG. 2C. This 8-10 centimeters or more separation reduces or prevents recirculation of dialyzed blood through the arteriovenous graft 202. The blood flow restrictor apparatus 214 can be placed or located in the arteriovenous graft 202 between such insertion points of the arterial cannula 108 and the venous cannula 110. The blood flow restrictor apparatus 214 permits the requisite high flow from the arterial cannula 108 and through the venous cannula 110 during dialysis, but restricts the blood flow through the fixed dimensions of the restrictor apparatus itself, during and between hemodialysis sessions. This reduces the complications associated with a high flow rate arteriovenous graft, such as high output heart failure, atrophy of the distal hand 212, or thrombosis secondary to venous hyperplasia or stenosis occurring either at the graft-vein anastomosis 222 or centrally in the subclavian or axillary veins, as discussed above.

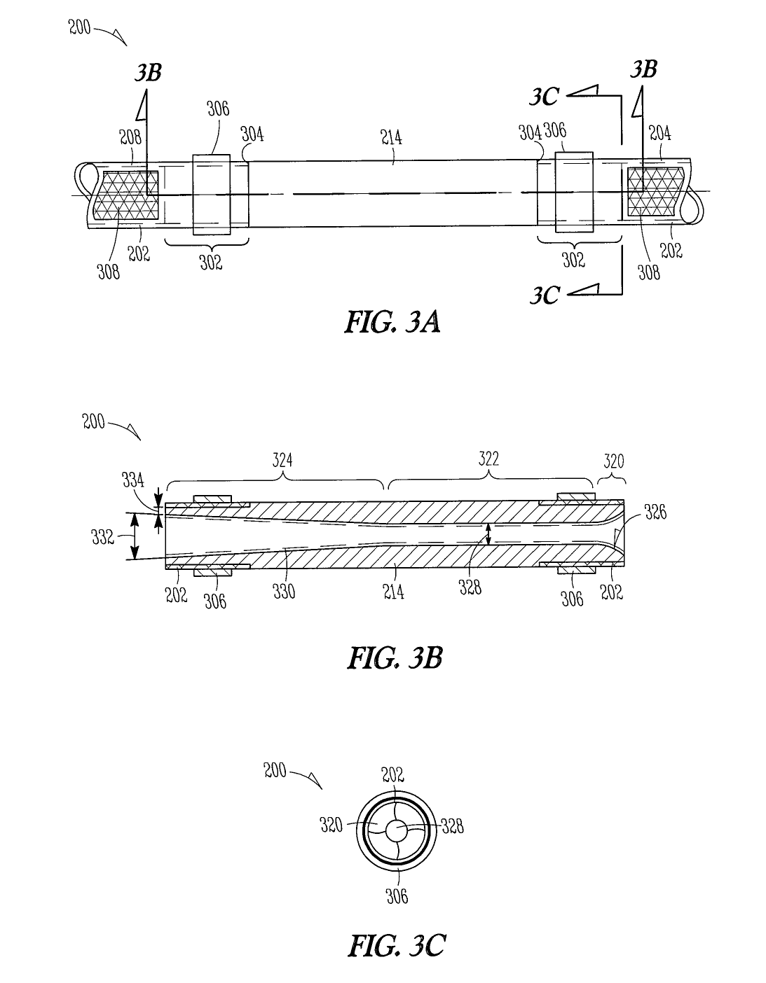

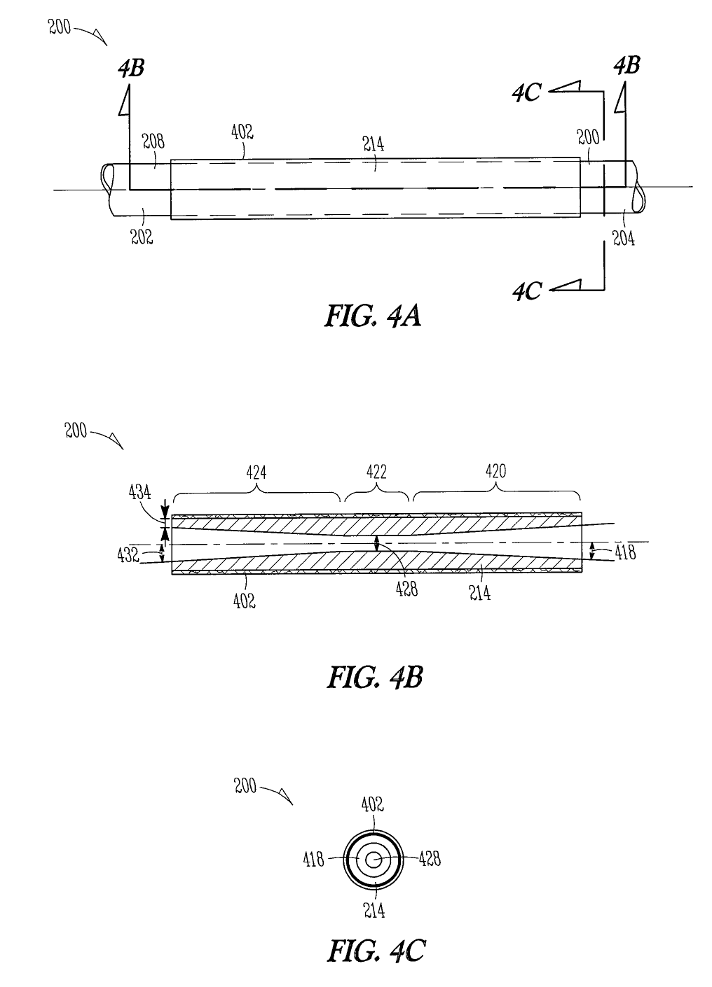

To prevent insertion of one or both of the arterial 108 or venous 110 cannula into the restrictor apparatus 214, the restrictor apparatus 214 itself can include a non-puncturable structure (see, e.g., FIGS. 3A-3B) or a rigid collar 402 or other puncture resistant covering can be disposed around an exterior of the restrictor apparatus 214 (see, e.g., FIGS. 4A-4B).

In certain examples, the arteriovenous graft 202 includes a tubular structure composed of or including a synthetic material, such as GORTEX™ manufactured by W.L. Gore & Associates, Inc. of Newark, Del. Additionally or alternatively, the arteriovenous graft 202 can include a woven or other self-sealing material made of any of a variety of one or more biocompatible materials, including biocompatible polymers, metals, alloys, or a combination thereof, such as polyester, polytetrafluoroethylene, polyethylene, polypropylene, polyurethane, silicone, stainless steel, titanium, or platinum, some of which are manufactured by Gish Biomedical, Inc. of Rancho Santa Margarita, Calif.

The human body may react to introduction of the synthetic materials of an arteriovenous graft 202. The body's reaction may include thrombus formation in or around the arteriovenous graft 202. While woven graft materials, such as GORTEX™, may not be recognized by the subject's body as a foreign body to the same degree as non-woven materials, woven materials may still experience some degree of body reaction, such as inflammation. For this reason, the arteriovenous grafts 202 can be made larger in interior diameter than what is needed to accommodate the dialysis machine's 106 (FIG. 1) about 400 cc/min requisite blood flow. This larger size, in turn, can result in high volume blood flow (e.g., 800-900 cc/min), which may further result in hyperplasia, among other things. Hyperplasia is a condition that may occur when the higher pressure/volume of the arterial flow crosses the boundary from the relatively non-compliant arteriovenous graft 202 to the more compliant outflow vein 210 at the venous anastomosis 222. The resultant intimae hyperplasia in the vein 210 adjacent to the anastomosis 222 may lead to progressive stenosis and eventually premature clotting and arteriovenous graft 202 occlusion. In addition to hyperplasia and stenosis, the large obligate shunted blood volumes may lead to an increased load on the heart and blood steal that results in poor circulation at the extremity beyond or distal to the arteriovenous graft 202.

The restrictor apparatus 214 comprises a size and shape that reduces the pressure and volume of blood flow through the arteriovenous graft 202 (e.g., by about 40-50%) generally without thrombus formation, and accordingly may reduce or eliminate the above discussed problems with hyperplasia, stenosis, increased heart load, or blood steal. Further, the restrictor apparatus 214 still allows adequate blood flow typically needed by the dialysis machine 106 during dialysis sessions (e.g., about 400 cc/min blood flow; however, in certain circumstances about 300 cc/min may suffice). In certain examples, but not by way of limitation, the arteriovenous graft 202 is about 5-6 inches long and about 6 millimeters in interior diameter outside the region of the restrictor apparatus 214. As shown, the implanted shape of the arteriovenous graft 202 between the subject's artery 206 and vein 210 can generally resemble a U-shape (i.e., make an approximate 180 degree change in direction). In one such example, the restrictor apparatus 214 is disposed on a generally straight leg portion of the U-shape. In another example, the restrictor apparatus 214 comprises a pliable (i.e., bendable) material and is disposed on a curved portion of the U-shape. As phantomly shown, the subject's vein 210 can be ligated 270 upstream of the graft-vein anastomosis 222.

Although the present examples focus on an arteriovenous graft system 200 subcutaneously implanted within a subject's arm 216 (see, e.g., FIG. 2B), the present subject matter is not so limited. The arteriovenous graft system 200 can alternatively be implanted in any suitable location of the subject's body 104 (FIG. 1). For instance, in certain examples, the arteriovenous graft system 200 can be implanted within a subject's leg 112 (FIG. 1).

FIG. 3A illustrates portions of an example of an arteriovenous graft system 200. The arteriovenous graft system 200 comprises an arteriovenous graft 202 and a restrictor apparatus 214. As shown, the restrictor apparatus 214 can comprise a structure separate from, but couplable to, the arteriovenous graft 202. In certain examples, the arteriovenous graft 202 comprises a tubular structure having an arterial end portion 204 and a venous end portion 208. The restrictor apparatus 214 can be interposed between the arterial 204 and venous 208 end portions and coupled to adjacent tubular arteriovenous graft 202 portions via reduced apparatus diameter portions 302. The reduced apparatus diameter portions 302 create a shoulder 304 on the restrictor apparatus 214 to which the arterial 204 and venous 208 end portions can abut against when the tubular graft portions 204, 208 are fitted over the reduced apparatus diameter portions 302. The arteriovenous graft 202 and the restrictor apparatus 214 can be securely coupled to one another via stainless steel clamps 306, such as those manufactured by Oetiker, Inc. of Marlette, Mich. Advantageously, clamp materials such as stainless steel and the like are durable, non-corrosive, and non-thrombogenic.

As discussed above, blood from the subject 104 (FIG. 1) flows from an artery 206 (FIG. 2C), through the shunted arteriovenous graft 202 and restrictor apparatus 214, and into a vein 210 (FIG. 2C). To connect the subject 104 to a dialysis machine 106, an arterial 108 and a venous 110 cannula (FIG. 2C) are inserted through the skin and into the arteriovenous graft 202. Blood is removed from the subject 104 through the arterial cannula 108, circulated through the dialysis machine 106, and returned to the subject 104 through the venous cannula 110. In certain examples, the arteriovenous graft 202 comprises a woven material 308 configured to be punctured by the cannulas 108, 110 and to self-seal upon their removal. In other examples, the arteriovenous graft 202 can include dedicated cannula injection portions, which include a self-sealing material, such as silicone or the like.

FIG. 3B is a side cross-sectional view taken along line 3B-3B of FIG. 3A and illustrates the interior structure of one example of an arteriovenous graft system 200. The arteriovenous graft system 200, according to this example, includes an arteriovenous graft 202 coupled to an intermediately disposed restrictor apparatus 214. The arteriovenous graft 202 is securely coupled to the restrictor apparatus 214 via one or more annular clamps 306, such as stainless-steel annular clamps. As shown, but as may vary, the restrictor apparatus 214 can include a side cross-sectional profile having three portions including a restrictor entry portion 320, a restrictor narrowed portion 322, and a restrictor exit portion 324. In another example, the restrictor apparatus 214 can include a side cross-sectional profile having two portions including a restrictor entry portion 320 and a restrictor exit portion 324. Each of the restrictor entry portion 320, the restrictor narrowed portion 322, and the restrictor exit portion 324, if present, have specified fixed internal dimensions (i.e., interior diameters and longitudinal lengths) based on one or more desired blood flow characteristics. Like most foreign objects introduced into a subject's body, it is advantageous to keep the exterior size of the restrictor apparatus 214 small.

In this example, the interior structure of the restrictor apparatus 214 includes a restrictor entry portion 320 having a radius of curvature, a constant diameter restrictor narrowed portion 322, and a gently tapered diverging restrictor exit portion 324. It is desirable to have a smooth transition between the arteriovenous graft 202 and the restrictor apparatus 214. A restrictor entry portion 320 having a large entry radius 326 reduces turbulence, which causes platelets in the blood to collide, and which can induce clot formation. To reduce or avoid turbulent blood flow, varying examples of the restrictor apparatus 214 comprise an entry having a radius of curvature of about 2 millimeters or more. As shown, the restrictor entry portion 320 tapers from (1) a diameter substantially similar to an interior diameter of the arteriovenous graft 202 on a first end of the restrictor entry portion 320 to (2) the diameter of the restrictor narrowed portion 322 on a second end of the restrictor entry portion 320.

The restrictor narrowed portion 322 is generally smooth and generally maintains a fixed and constant diameter 328 along its length. The generally smooth finish of the restrictor narrowed portion 322 helps to prevent thrombosis by not encouraging turbulent blood flow. A longer restrictor narrowed portion 322 will generally further reduce blood flow, but should not be so long as to encourage clotting. In certain examples, the restrictor narrowed portion 322 includes a length of between 1-100 millimeters, such as at least about 25 millimeters. In certain examples, the effective interior diameter of the restrictor narrowed portion 322 is at least about 1.5 millimeters. In certain other examples, the effective interior diameter of the restrictor narrowed portion 322 is at least about 2.5 millimeters, which is believed to stop high viscous shear rates and to successfully reduce the flow of blood through the arteriovenous graft system 200.

To inhibit thrombus formation, the restrictor apparatus 214 can comprise a coating of a biologically active layer 330 (e.g., an anti-thrombogenic coating), such as that manufactured by Carmeda of Upplands Vasby, Sweden, which effectively reduces the interior diameter 328 of the restrictor narrowed portion 322. Thus, in certain examples, the pre-coating interior diameter 328 of the restrictor narrowed portion 322 is about 2.8-3.0 millimeters, such that when the biologically active layer 330 is taken into account, the effective interior diameter 328 of the restrictor narrowed portion is about 2.5 millimeters or more. The biologically active layer 330 can be applied to the surface of the restrictor narrowed portion 322 by coating, spraying, dipping, or vapor deposition. Such layer 330 can extend along the linear length as phantomly shown in FIG. 3B, or be localized to a particular area.

The restrictor exit portion 324 is shown gently tapered having an exit angle 332. Computer simulation indicates that an exit angle 332 of about 6 degrees or less advantageously inhibits or prevents blood flow separation or flow turbulence. As shown, the restrictor exit portion 324 diverges from the diameter 328 of the restrictor narrowed portion 322 on a first end to a diameter that is substantially similar to the interior diameter of the arteriovenous graft 202 on a second end. In certain examples, a step 334 of about 0.5 millimeters or less can exist at the exit of the restrictor apparatus 214 so that there is essentially no discontinuity between the exit portion 324 of the restrictor and the interior diameter of the arteriovenous graft 202.

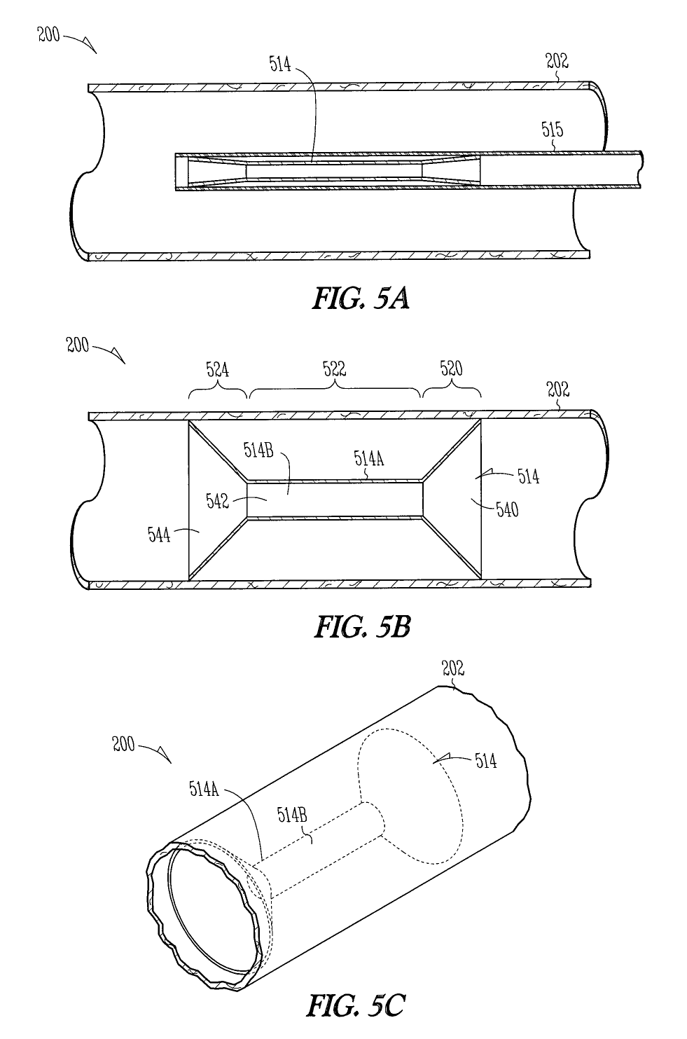

Together, in at least one example, the restrictor entry portion 320, the restrictor narrowed portion 322, and the restrictor exit portion 324 decrease the dynamic pressure and volume of blood flow passing through the arteriovenous graft system 200. This lessens the blood steal from a limb 212 (FIG. 2B) peripheral to the arteriovenous graft system 200 and reduces the blood flow loads on the heart and veins, all without affecting needed dialysis flow rates and without encouraging clotting. The amount of flow restriction provided by the restrictor apparatus 214 is dependent on the interior diameter and length of the apparatus, such as the interior diameter and length of the restrictor narrowed portion 322. For instance, a longer restrictor narrowed portion 322 generally results in greater flow restriction, but may result in clotting if too long. On the other hand, a shorter restrictor narrowed portion 322 generally results in less flow restriction and can therefore be less effective in reducing blood steal (see, e.g., FIG. 5). A greater restrictor narrowed portion diameter 328 generally results in less clotting, but also less restriction and more blood steal. Advantageously, the separate structure restrictor apparatus 214 illustrated in FIGS. 3A-3C can be used with a conventional vascular access graft, such as by retrofitting the restrictor apparatus 214 into an intermediate portion of an existing arteriovenous graft 202 that has been cut into two pieces. Alternatively, the separate structure restrictor apparatus 214 can be disposed (e.g., slid) within a conventional vascular access graft.

FIG. 3C is a transverse cross-section along line 3C-3C of FIG. 3A and illustrates the varying diameters of one example of an arteriovenous graft system 200. Taken at an outermost end of a reduced diameter portion 302 (FIG. 3A), the cross-section shown in FIG. 3C shows an annular clamp 306 encircling a tubular arteriovenous graft 202 and the tapered restrictor entry portion 320. As shown, the restrictor entry portion 320 tapers to an interior diameter 328 of the restrictor narrowed portion 322. While FIGS. 3A-3C illustrate a traverse cross-section of the arteriovenous graft system 200 having a circular configuration, the traverse cross-section can also be oval or some other configuration.

FIG. 4A illustrates portions of another example of an arteriovenous graft system 200. In this example, the arteriovenous graft system 200 comprises an arteriovenous graft 202 and an integral restrictor apparatus 214. Unlike the restrictor apparatus 214 of FIGS. 3A-3C, the restrictor apparatus 214 of FIGS. 4A-4C together with the arteriovenous graft 202 comprise a unitary construction. The restrictor apparatus 214 can be encircled or surrounded, at least in part, by a relatively non-penetrable (i.e., non-puncturable) collar 402. This prevents cannula 108, 110 (FIG. 2C) insertions into the restrictor apparatus and helps permit a caregiver to be able to palpate the restrictor to determine its position. In certain examples, the collar 402 comprises a rigid biocompatible material, such as a biocompatible metal (e.g., titanium or stainless-steel) or a biocompatible plastic.

FIG. 4B is a side cross-sectional view taken along line 4B-4B of FIG. 4A and illustrates the interior structure of another example of an arteriovenous graft system 200. The arteriovenous graft system 200, in this example, includes an arteriovenous graft 202 integrated with a restrictor apparatus 214. As shown, the restrictor apparatus 214 can include a side cross-sectional profile that includes a restrictor entry portion 420, a restrictor narrowed portion 422, and a restrictor exit portion 424. Each of the restrictor entry portion 420, the restrictor narrowed portion 422, and the restrictor exit portion 424 have specified fixed internal dimensions (i.e., interior diameters and longitudinal lengths), which can be established based on one or more desired blood flow characteristics. For instance, the arteriovenous graft system 200 can include varying interior dimensions in the vicinity of the restrictor apparatus 214 such that the walls are thicker at the restrictor entry portion 420, the restrictor narrowed portion 422, and the restrictor exit portion 424 than at the arterial 204 and venous 208 end portions of the arteriovenous graft 202 (FIG. 4A).

In this example, the interior structure of the restrictor apparatus 214 includes a gently tapered converging restrictor entry portion 420, a constant diameter restrictor narrowed portion 422, and a gently tapered diverging restrictor exit portion 424. It is believed to be desirable to have a smooth transition between the interior diameter of the arteriovenous graft 202 and that of the restrictor apparatus 214. A restrictor entry portion 420 having as large (or near as large) as entry radius 326 (FIG. 3B) as possible may reduce turbulence, which causes platelets in the blood to collide and may induce clot formation. To avoid turbulent blood flow, in certain examples, the restrictor apparatus 214 includes an entry having a radius of curvature of at least about 2 millimeters. As another example, FIG. 4B shows an example in which the restrictor entry portion 420 can include a converging tapered entry angle 418 of about 6 degrees or less. The restrictor narrowed portion 422 is generally smooth and maintains a fixed and constant diameter 428 along its length. The generally smooth finish of the restrictor narrowed portion 422 helps to prevent thrombosis by not encouraging turbulent blood flow. A longer restrictor narrowed portion 422 further reduces blood flow; however, the restrictor narrowed portion 422 should not be so long as to reduce flow to an extent that encourages clotting. In certain examples, the restrictor narrowed portion 422 comprises a length between 1-100 millimeters, such as at least about 25 millimeters. In certain examples, the effective interior diameter of the restrictor narrowed portion 322 is at least about 1.5 millimeters. In certain other examples, the effective interior diameter of the restrictor narrowed portion 322 is at least about 2.5 millimeters, which is expected to stop high viscous shear rates and to successfully reduce the flow of blood through the arteriovenous graft system 200.

The restrictor exit portion 424 is shown gently tapered having an exit angle 432. An exit angle 432 of about 6 degrees or less advantageously prevents blood flow separation and flow turbulence. As shown, the restrictor exit portion 424 diverges from the diameter 428 of the restrictor narrowed portion 422 on a first end to a diameter substantially similar to the interior diameter of the arteriovenous graft 202 on a second end. A step 434 of about 0.5 millimeters or less can exist at the exit of the restrictor apparatus 214 so that there is essentially no discontinuity between the restrictor and the interior diameter of the arteriovenous graft 202.

In certain examples, the restrictor entry portion 420, the restrictor narrowed portion 422, and the restrictor exit portion 424 decrease the dynamic pressure and volume of blood flow passing through the arteriovenous graft system 200. This lessens the blood steal from a peripheral limb 212 (FIG. 2B) and reduces the blood flow load on the heart and veins, all without affecting needed dialysis flow rates and without encouraging clotting. The amount of flow restriction provided by the restrictor apparatus 214 depends on its interior diameter and length, such as the interior diameter and length of the restrictor narrowed portion 322. For instance, a longer narrowed portion 422 will further reduce flow, but may result in clotting if too long. A greater diameter 428 of the narrowed portion will result in less clotting, but also less flow restriction.

FIG. 4C is a transverse cross-section along line 4C-4C of FIG. 4A and illustrates the varying diameters of one example of an arteriovenous graft system 200. Taken at an end of the restrictor apparatus 214, the cross-section shown in FIG. 4C shows a collar 402 about the walls of the restrictor apparatus 214 and the tapered restrictor entry portion 420. As shown, the restrictor entry portion 420 tapers to an interior diameter 428 of the restrictor narrowed portion 422. While FIGS. 4A-4C illustrate a traverse cross-section of the arteriovenous graft system 200 having a circular configuration, the traverse cross-section can also be oval or some other configuration.

In some examples, a restrictor apparatus 214 can be inserted within an existing arteriovenous graft 202, which is already implanted within a subject's body. In some examples, the restrictor apparatuses 214 described herein can be inserted at desired endovascular locations other than within an arteriovenous graft 202.

For instance, FIGS. 5A-5C illustrate an example of insertion of a shape memory restrictor apparatus 514 into an arteriovenous hemodialysis access, such as an arteriovenous graft 202. The “shape memory” property permits the apparatus 514 to “remember” a previous shape. For example, the shape memory restrictor apparatus 514 can be compressed or otherwise deformed (e.g., compressed within a sleeve), and can then return toward or regain its pre-deformation shape when uncompressed or otherwise released (e.g., when the sleeve is removed). The shape memory apparatus 514 can include a generally tubular wall 514A defining a lumen 514B therethrough. Although described below with respect to use in an arteriovenous graft 202, it is contemplated in other examples that the shape memory apparatus 514 can be used with other arteriovenous hemodialysis accesses, such as an arteriovenous fistula.

In some examples, a compressed shape memory restrictor apparatus 514 can be endovascularly inserted into the already-implanted arteriovenous graft 202. In certain examples, the shape memory restrictor apparatus 514 is stent-like in configuration. In some examples, the shape memory restrictor apparatus 514 can be formed from one or more materials including, but not limited to, a shape memory metal, such as Nitinol. In some examples, the shape memory restrictor apparatus 514 can include a substantially impermeable coating, membrane, or other material, such as, for instance, Dacron or polytetrafluoroethylene (PTFE). The substantially impermeable material, in some examples, can stretch when the shape memory restrictor apparatus 514 is expanded, as described herein, to define a substantially fluid impermeable wall to perform blood flow restriction, as described herein. The shape memory restrictor apparatus 514 can include a shape memory metal with a substantially impermeable material coating, sheath, or surface, such as to inhibit or prevent blood or other fluids from passing through the generally tubular wall 514A of the shape memory restrictor apparatus 514.

In an example, the compressed shape memory restrictor apparatus 514 can be compressed within a retractable sleeve 515, such as for delivery to and/or deployment at a desired location. In an example, the compressed shape memory restrictor apparatus 514 can be delivered to a location within the implanted arteriovenous graft 202, such as by using an intravascular delivery catheter. Once at the desired implant location, the shape memory restrictor apparatus 514 can be released or otherwise deployed, such as to allow the shape memory restrictor apparatus 514 to uncompress and take a desired implanted shape within the arteriovenous graft 202. In an example, the shape memory restrictor apparatus 514 can be released, such as by retracting the retractable sleeve 515 and allowing the shape memory restrictor apparatus 514 to assume the desired shape. In an example, the shape memory restrictor apparatus 514 is capable of expanding to a maximum diameter that is larger than a diameter of the arteriovenous graft 202. This creates a frictional engagement of the outer diameter of the shape memory restrictor apparatus 514 and the inner diameter of the arteriovenous graft 202 when the shape memory restrictor apparatus 514 is released therein. In an example, the shape memory restrictor apparatus 514 is capable of expanding to a non-constricting maximum diameter in which a diameter of the ends of the restrictor apparatus 514 are larger than a diameter of the arteriovenous graft 202 when the restrictor apparatus 514 is unconstrained. This can be used to create a frictional engagement of the outer diameter of the ends of the shape memory restrictor apparatus 514 and the inner diameter of the arteriovenous graft 202 when the restrictor apparatus 514 is released therein.

In an example, the uncompressed shape memory restrictor apparatus 514 can include an entry portion 520. The entry portion 520 can include a convergent first lumen portion 540 that tapers to substantially match an interior diameter of an arterial portion of the arteriovenous graft 202. In an example, the uncompressed shape memory restrictor apparatus 514 includes an exit portion 524. The exit portion 524 can include a divergent second lumen portion 544 that tapers to substantially match an interior diameter of a venous portion of the arteriovenous graft 202. In an example, the uncompressed shape memory restrictor apparatus 514 can include an intermediate portion 522 between the entry portion 520 and the exit portion 524. In an example, the intermediate portion 522 can include a substantially cylindrical third lumen portion 542.

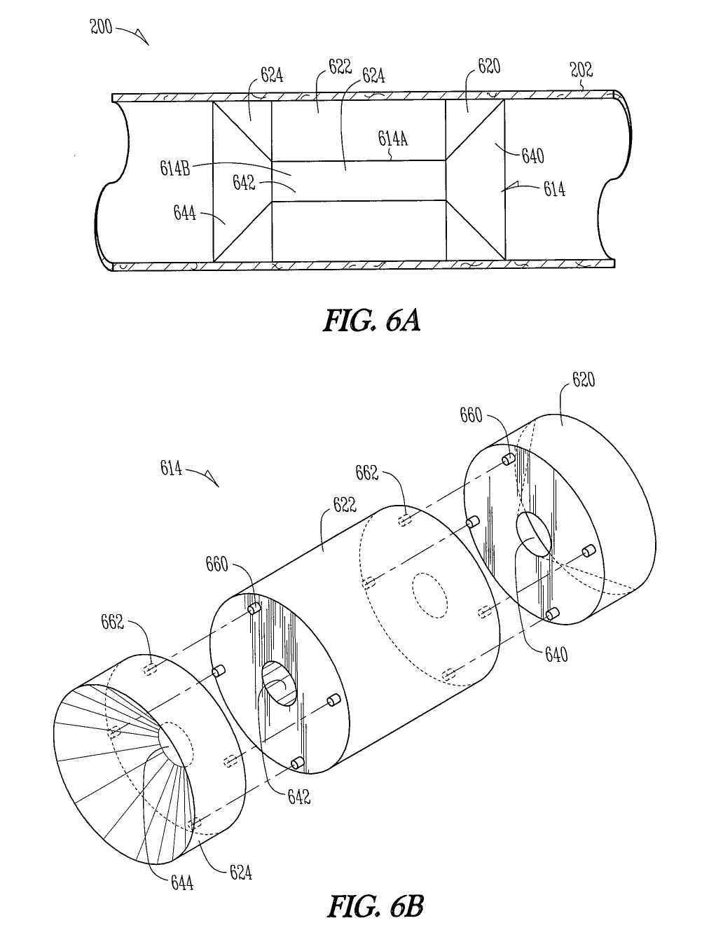

FIGS. 6A and 6B illustrate an example shape memory restrictor apparatus 614, in accordance with an embodiment. When implanted, in an example, the shape memory restrictor apparatus 614 can be generally similar to the shape memory restrictor apparatus 514 described above. However, in some examples, the shape memory restrictor apparatus 614 can include more than one piece. The pieces can be inserted separately and attached within the subject, or the pieces can be fused or otherwise attached together before implantation and then implanted. The shape memory restrictor apparatus 614, in some examples, can include a shape memory blood restrictor apparatus 614 for implantation within an arteriovenous hemodialysis access, such as an arteriovenous graft 202. Although described with respect to use in an arteriovenous graft 202, it is contemplated in other examples that the shape memory apparatus 614 can be used with other arteriovenous hemodialysis accesses, such as an arteriovenous fistula. In some examples, the shape memory restrictor apparatus 614 can be formed from similar materials as those described above with respect to the shape memory restrictor apparatus 514.

In some examples, the shape memory restrictor apparatus 614 can include a two-piece shape memory restrictor apparatus 614, such as having a first piece 620 including an entry portion and a second piece 624 including an exit portion. In some examples, the shape memory restrictor apparatus 614 can include a third piece 622, such as including an intermediate portion. In some examples, the shape memory restrictor apparatus 614 can include pieces in addition to (e.g., and in accordance with) the first, second, and third pieces 620, 624, 622 described herein.

In an example, the pieces of the shape memory restrictor apparatus 614 can be individually compressed and retained within one or more retractable sleeves for endovascular (for instance, intravenous) insertion into the arteriovenous graft 202. The pieces of the shape memory restrictor apparatus 614 can each be retained within a separate retractable sleeve, or two or more pieces can be retained in a single retractable sleeve. The pieces of the shape memory restrictor apparatus 614 can then be endovascularly inserted into the implanted arteriovenous graft 202, for instance, using a single delivery catheter or other delivery technique. In some examples, at least two of the pieces of the shape memory restrictor apparatus 614 can be endovascularly inserted into the arteriovenous graft 202 using different delivery catheters. Once inserted within the arteriovenous graft 202, the sleeves can be retracted, such as to deploy and permit decompression of each of the pieces of the shape memory apparatus restrictor 614. In an example, mating or other engagement features 660, 662, such as generally depicted in FIG. 6B, can be engaged to each other to attach the pieces of the shape memory restrictor apparatus 614. In some examples, the engagement features 660, 662 can include, but are not limited to, one or more mating hooks or clasps, magnets, mating detents, pins, docking mechanisms, or adhesive surfaces. In an example, the shape memory restrictor apparatus 614 need not include any engagement features; the pieces of the shape memory restrictor apparatus 614 can be held together through mutual and adjacent frictional engagement with the arteriovenous graft 202 when deployed. In an example, the pieces of the shape memory restrictor apparatus 614 can be fused together or otherwise attached before implantation, and then compressed and endovascularly inserted within the arteriovenous graft 202, such as by using a retractable sleeve and delivery catheter or another delivery technique.

Once deployed in the desired location within the arteriovenous graft 202, in some examples, the pieces of the shape memory restrictor apparatus 614 can take desired shapes (or “remember” and return toward their intended shapes). In an example, the uncompressed first piece 620 forms an entry portion of the shape memory restrictor apparatus 614 that includes a convergent first lumen 640 that tapers to substantially match an interior diameter of an arterial portion of the arteriovenous graft 202. In an example, the uncompressed second piece 624 forms an exit portion of the shape memory restrictor apparatus 614 that includes a divergent second lumen 644 that tapers to substantially match an interior diameter of a venous portion of the arteriovenous graft 202. In an example, the uncompressed third piece 622 forms an intermediate portion of the shape memory restrictor apparatus 614 between the first piece 620 and the second piece 624. In an example, the third piece 622 can include a substantially cylindrical third lumen 642. When attached, the pieces of the shape memory restrictor apparatus 614 can, in an example, form a generally continuous tubular wall 614A defining a lumen 614B therethrough.

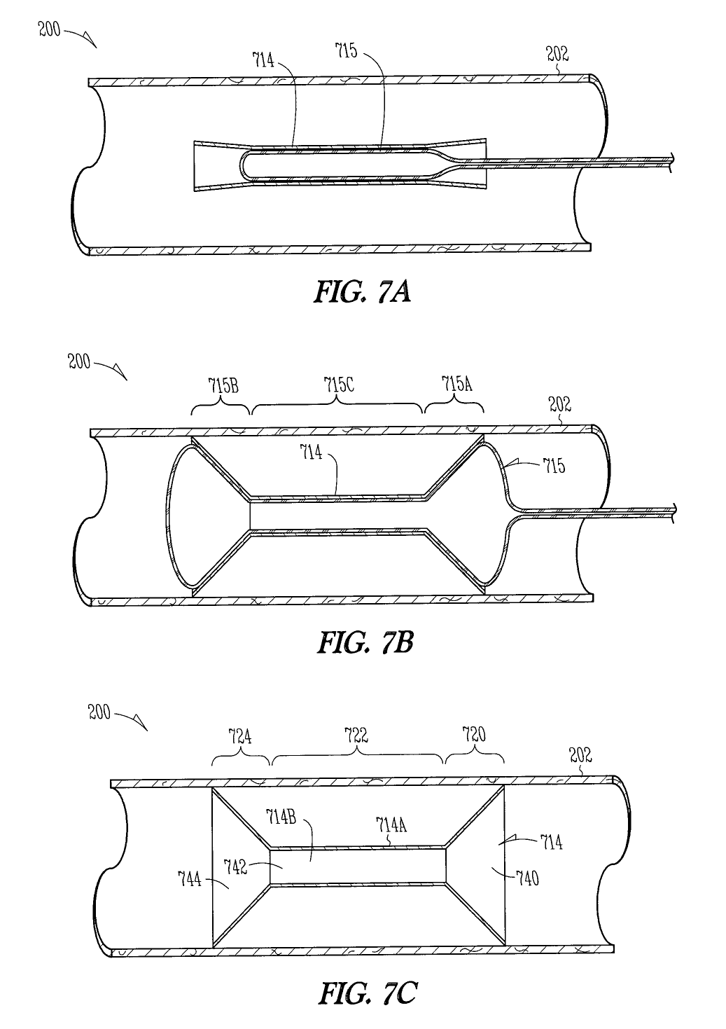

FIGS. 7A-7C illustrate an example of insertion of an example of a restrictor apparatus 714, in the form of a moldable stent, into an arteriovenous hemodialysis access, such as an arteriovenous graft 202. Although described with respect to use in an arteriovenous graft 202, it is contemplated in other examples that the stent 714 can be used with other arteriovenous hemodialysis accesses, such as an arteriovenous fistula. In an example, a deflated balloon 715 and the moldable stent 714 can be endovascularly inserted within an arteriovenous graft 202, for instance, using a delivery catheter or a similar delivery technique. In some examples, the stent 714 can be formed from a moldable material, such as metal. In an example, the stent 714 can be formed from stainless steel. In an example, the stent 714 can include a layer of a substantially impermeable material, such as, but not limited to, Dacron or PTFE. The substantially impermeable material layer, in some examples, can stretch when the stent 714 is expanded, as described below, to define a substantially fluid impermeable wall to perform blood flow restriction, as described herein.

Once deployed at a desired location, for instance, within the arteriovenous graft 202, the balloon 715 can be inflated within the moldable stent 714. In an example, the balloon 715 can include an inflated shape (see, e.g., FIG. 7B) having a first section 715A at a first end and a second section 715B at a second end. The first section 715A, in an example, can be substantially conical and converging from the first end toward the second end. The second section 715B can be substantially conical and converging from the second end toward the first end. In a further example, the inflated shape of the balloon 715 includes a third section 715C between the first section 715A and the second section 715B, the third section 715C being substantially cylindrical.

In an example, inflating the balloon 715 within the moldable stent 714 forces the moldable stent 714 outward and take a shape similar to that of the inflated balloon 715. In effect, a wall 714A of the stent 714 substantially assumes the shape of an outer surface of the balloon 715 to define a lumen 714B, which is essentially a “negative shape” of the balloon 715. In an example, the stent 714 can be expanded into engagement with an interior surface of the arteriovenous graft 202, such as to frictionally engage the stent 714 with the arteriovenous graft 202. In other examples, the stent 714 can include mating or other engagement features, such as for mating or otherwise engaging with the arteriovenous graft 202, such as at corresponding engagement features of the arteriovenous graft 202.

In an example, once the stent 714 is positioned and shaped, the balloon 715 can be deflated and removed from within the moldable stent 714. The moldable stent 714 can maintain its shape similar to that of the inflated balloon 715. In an example, the stent 714 can be shaped to form an entry portion 720 that can include a convergent first lumen 740 that tapers to substantially match an interior diameter of an arterial portion of the arteriovenous graft 202. In an example, the stent 714 can be shaped to form an exit portion 724 that includes a divergent second lumen 744 that tapers to substantially match an interior diameter of a venous portion of the arteriovenous graft 202. In another example, the stent 714 can be shaped to form an intermediate portion 722 between the entry portion 720 and the exit portion 724. In an example, the intermediate portion 722 can include a substantially cylindrical third lumen 742. When shaped, in an example, the stent 714 can form a generally continuous tubular wall 714A defining a lumen 714B therethrough. When inserted within the arteriovenous graft 202, in an example, the stent 714, formed such as described above, can provide a restrictor apparatus to function in a manner similar to examples of restrictor apparatuses described herein.

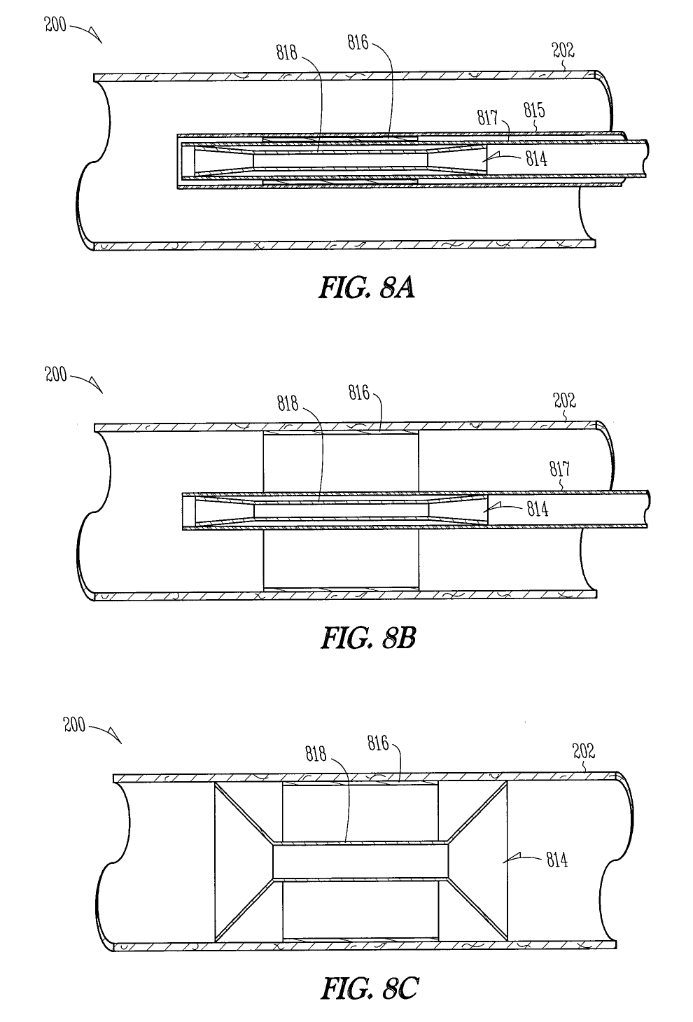

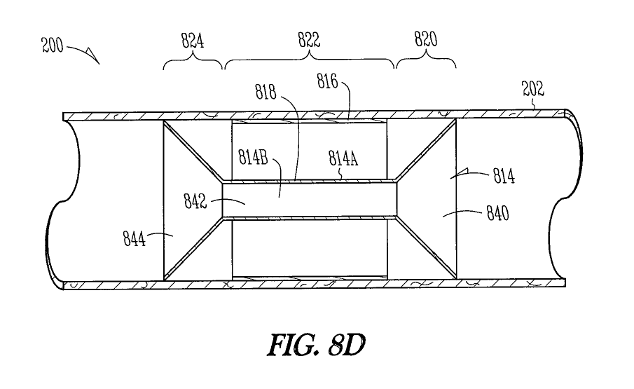

FIGS. 8A-8D illustrate an example of insertion of an example of a restrictor apparatus 814 into an arteriovenous hemodialysis access, such as an arteriovenous graft 202. Although described with respect to use in an arteriovenous graft 202, it is contemplated in other examples that the restrictor apparatus 814 can be used with other arteriovenous hemodialysis accesses, such as an arteriovenous fistula. In an example, an outer piece 816 of a blood flow restrictor apparatus 814 is endovascularly inserted into the arteriovenous graft 202. In an example, the outer piece 816 can be formed from a shape memory material that can be compressed and retained within a first retractable sleeve 815 for delivery using a catheter or other delivery technique. When uncompressed or otherwise deployed, the outer piece 816 can include a first diameter and a first length. In an example, the outer piece 816 can form a substantially cylindrical tube in which the first diameter is substantially equal to an interior diameter of the arteriovenous graft 202. In an example, the outer piece 816 can be capable of expanding to include a first diameter that is larger than the interior diameter of the arteriovenous graft 202. This allows frictional engagement of the outer piece 816 with the arteriovenous graft 202.

In an example, an inner piece 818 of the blood flow restrictor apparatus 814 can be endovascularly inserted within the outer piece 816. In an example, the inner piece 818 can be formed from a shape memory material that can be compressed and retained within a second retractable sleeve 817 such as for delivery using a catheter or other delivery technique. When allowed to decompress or otherwise deployed, the inner piece can include a shaped inner profile including a convergent first portion 820 that tapers to substantially match an interior diameter of an arterial portion of the arteriovenous graft 202 and a divergent second portion 824 that tapers to substantially match an interior diameter of a venous portion of the arteriovenous graft 202. In an example, the shaped profile of the inner piece 818 includes a third portion 822 between the first portion 820 and the second portion 824, the third portion 822 including a substantially cylindrical lumen portion.

In an example, a distal end of the inner piece 818 can be attached to a distal end of the outer piece 816. In an example, the distal end of the inner piece 818 can be attached to the distal end of the outer piece 816, such as by attaching an engagement feature of one of inner and outer pieces 818, 816 with a mating engagement feature of the other of the inner and outer pieces 818, 816. In some examples, the engagement features can include, but are not limited to, mating hooks or clasps, magnets, mating detents, pins, docking mechanisms, or adhesive surfaces. In an example, the distal end of the inner piece 818 can be frictionally engaged to the distal end of the outer piece 816.

In an example, a proximal end of the inner piece 818 can be attached to a proximal end of the outer piece 816. In an example, the proximal end of the inner piece 818 can be attached to the proximal end of the outer piece 816 by attaching an engagement feature of one of inner and outer pieces 818, 816 with a mating engagement feature of the other of the inner and outer pieces 818, 816. In some examples, the engagement features can include, but are not limited to, mating hooks or clasps, magnets, mating detents, pins, docking mechanisms, or adhesive surfaces. In an example, the proximal end of the inner piece 818 can be frictionally engaged to the proximal end of the outer piece 816.

In this way, in an example, the outer and inner pieces 816, 818 can be joined to form a substantially unitary structure with the inner piece 818 of the blood flow restrictor apparatus 814 forming a generally continuous tubular wall 814A defining a lumen 814B therethrough. When inserted within the arteriovenous graft 202, in an example, the blood flow restrictor apparatus 814, formed as described above, can function in a manner similar to examples of restrictor apparatuses described herein.

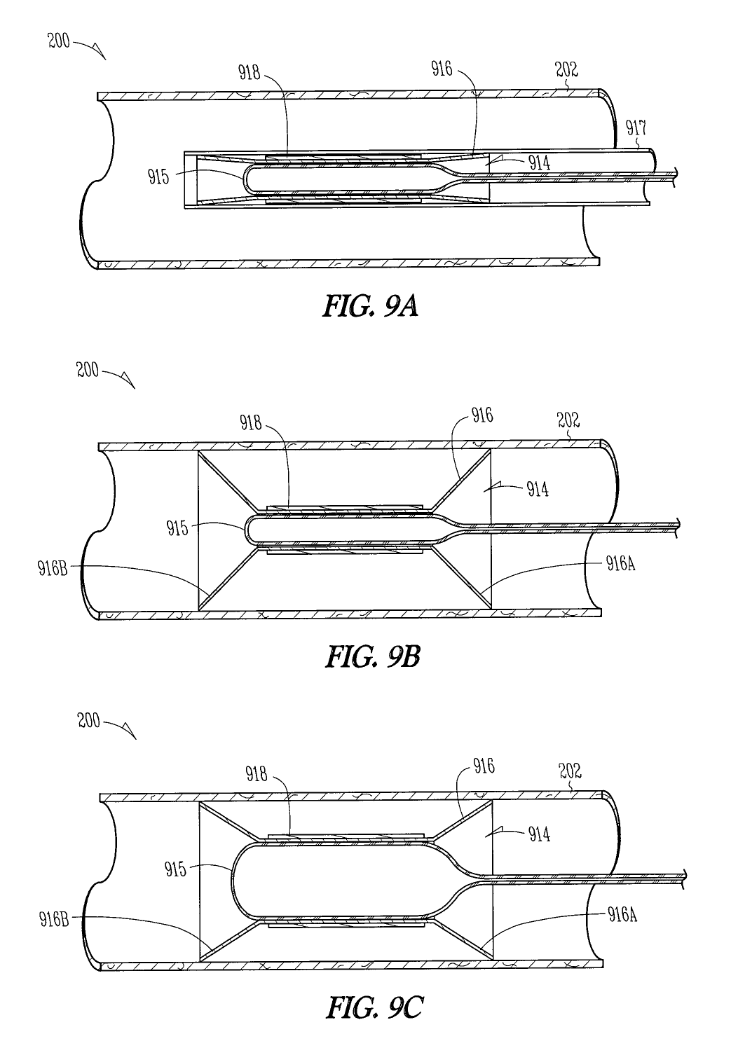

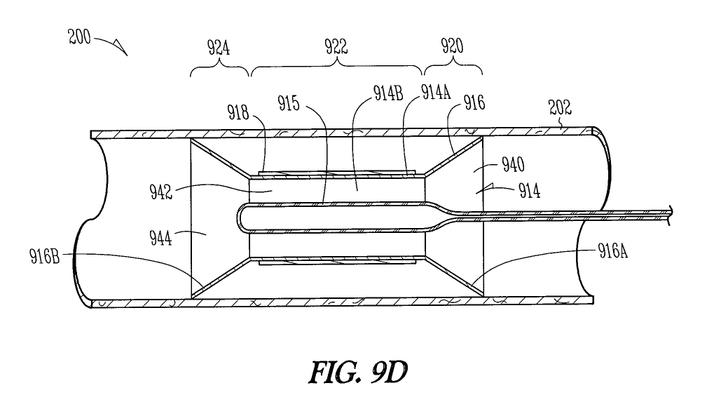

FIGS. 9A-9D illustrate an example of insertion of an example of a restrictor apparatus 914 into an arteriovenous hemodialysis access, such as an arteriovenous graft 202. Although described with respect to use in an arteriovenous graft 202, it is contemplated in other examples that the restrictor apparatus 914 can be used with other arteriovenous hemodialysis accesses, such as an arteriovenous fistula. In an example, a deflated balloon 915, a shape memory apparatus 916, and a moldable stent 918 or other such moldable apparatus can be endovascularly inserted within an arteriovenous graft 202, for instance, using a delivery catheter or a similar delivery technique. In certain examples, the shape memory apparatus 916 can be stent-like in configuration. In some examples, the shape memory apparatus 916 can be formed from one or more materials including, but not limited to, a shape memory metal, such as Nitinol. In some examples, the shape memory apparatus 916 can include a substantially impermeable coating, membrane, or other material, such as, for instance, Dacron or polytetrafluoroethylene (PTFE). The substantially impermeable material, in some examples, can stretch when the shape memory apparatus 916 is expanded, such as described herein, such as to define a substantially fluid impermeable wall to perform blood flow restriction, such as described herein. The shape memory apparatus 916 can include a shape memory metal with a substantially impermeable material coating, sheath, or surface, such as to inhibit or prevent blood or other fluids from passing through a generally tubular wall 914A of the restrictor apparatus 914. In some examples, the moldable stent 918 can be formed from a moldable material, such as metal. In an example, the moldable stent 918 can be formed from stainless steel.

In an example, a compressed restrictor apparatus 914 can be compressed within a retractable sleeve 917, such as for delivery to a desired location. In an example, the compressed restrictor apparatus 914 can be delivered to a location within the implanted arteriovenous graft 202. In an example, the compressed restrictor apparatus 914 can include the moldable stent 918 disposed around the compressed shape memory apparatus 916, with the deflated balloon 915 disposed within each of the moldable stent 918 and the compressed shape memory apparatus 916.

Once at the desired implant location, the restrictor apparatus 914 can be released, such as to allow the shape memory apparatus 916 to uncompress and take a desired shape within the arteriovenous graft 202. In an example, the shape memory apparatus 916 can be released, such as by retracting the retractable sleeve 917 and allowing portions of the shape memory apparatus 916 unconstrained by the moldable stent 918 to assume the desired shape. In an example, ends 916A, 916B of the shape memory apparatus 916, which extend outwardly from the moldable stent 918, are capable of expanding to a maximum diameter in which diameters of ends 916A, 916B of the shape memory apparatus 916 are larger than a diameter of the arteriovenous graft 202 when the shape memory apparatus 916 is unconstrained. This can be used to create a frictional engagement of the outer diameter of the ends 916A, 916B of the shape memory apparatus 916 and the inner diameter of the arteriovenous graft 202 when the shape memory apparatus 916 is released therein.

Once deployed at a desired location, for instance, within the arteriovenous graft 202, the balloon 915 can be inflated within the moldable stent 918. In an example, the balloon 915 can include a substantially cylindrical inflated shape. In an example, inflating the balloon 915 within the moldable stent 918 forces the moldable stent 918 and the portion of the shape memory apparatus 916 to expand and take a substantially cylindrical shape, such as of a desired blood flow restrictive inner diameter.

In an example, once the restrictor apparatus 914 is positioned and shaped, the balloon 915 can be deflated and removed from within the restrictor apparatus 914. In an example, the restrictor apparatus 914 can be shaped to form an entry portion 920 that can include a convergent first lumen 940 that tapers to substantially match an interior diameter of an arterial portion of the arteriovenous graft 202. In an example, the restrictor apparatus 914 can be shaped to form an exit portion 924 that includes a divergent second lumen 944 that tapers to substantially match an interior diameter of a venous portion of the arteriovenous graft 202. In another example, the restrictor apparatus 914 can be shaped to form an intermediate portion 922 between the entry portion 920 and the exit portion 924. In an example, the intermediate portion 922 can include a substantially cylindrical third lumen 942. When shaped, in an example, the restrictor apparatus 914 can form a generally continuous tubular wall 914A defining a lumen 914B therethrough. When inserted within the arteriovenous graft 202, in an example, the restrictor apparatus 914, formed such as described above, can provide a restrictor apparatus to function in a manner similar to examples of restrictor apparatuses described herein.

As particularly described with respect to the examples herein, restrictor apparatuses can be implemented within existing arteriovenous grafts 202 already implanted within a subject. However, in some examples, the restrictor apparatuses described above can be positioned within the arteriovenous graft 202 before implanting the arteriovenous graft 202, such as into a human or animal subject.

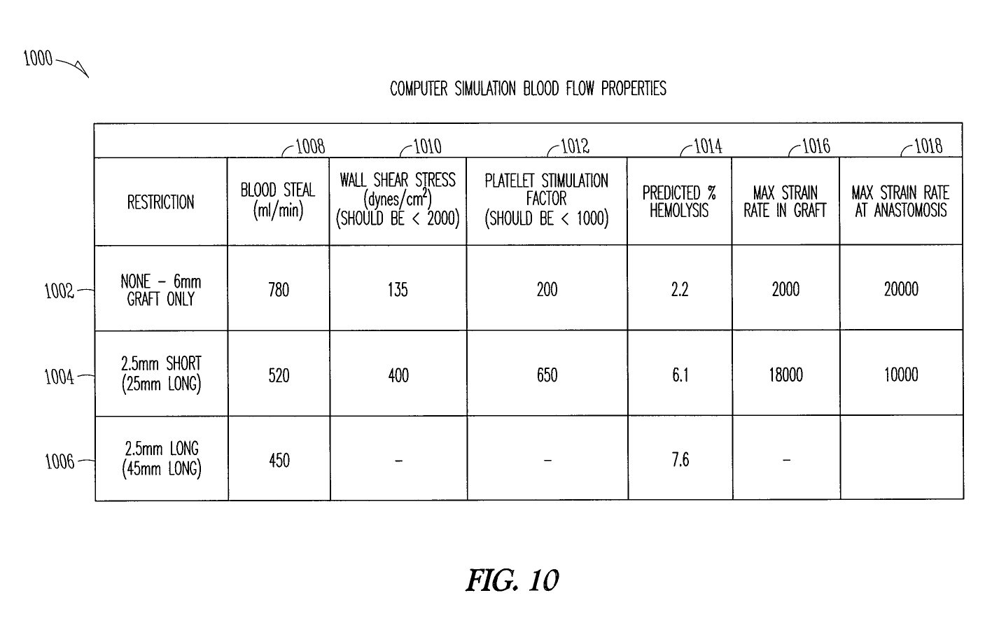

FIG. 10 is a summary chart 1000 from a computer simulation comparing the simulated blood flow properties within a subject 104 (FIG. 1) and in an arteriovenous graft 202 or an arteriovenous graft system 200 (FIG. 2B) (including an arteriovenous graft 202 and a restrictor apparatus 214 (FIG. 2B)) implanted within the subject. Line 1002 of the summary chart 1000 lists the simulated blood flow properties occurring within the subject 104 and in the arteriovenous graft 202 (which does not include a restrictor apparatus 214). Lines 1004 and 1006 of the summary chart 1000 list the simulated blood flow properties occurring within the subject 104 and in arteriovenous graft systems 200 including restrictor narrowed portions 322 (see, e.g., FIG. 3B) of 25 millimeters and 45 millimeters in length, respectively. The computer simulation according to this example assumes a graft tubular interior diameter of about 6 millimeters, an effective interior diameter 328 of the restrictor narrowed portion 322 of about 2.5 millimeters, and a divergent exit angle 332 of about 6 degrees with respect to a coaxial central axis of the restrictor apparatus 214.

As shown, the peripheral blood steal 1008 occurring within the subject 104 implanted with a non-restrictive arteriovenous graft 202 is simulated as being much greater than the peripheral blood steal 1008 occurring within the subject 104 implanted with a restrictive arteriovenous graft system 200. More specifically, the peripheral blood steal 1008 occurring within the subject 104 implanted with the arteriovenous graft system 200 including a 25 millimeter long restrictor narrowed portion 322 was simulated as being about 33% less than the peripheral blood steal 1008 occurring within the subject 104 implanted with the non-restrictive arteriovenous graft 202; while the blood steal 1008 within the subject 104 implanted with the arteriovenous graft system 200 including a 45 millimeter long restrictor narrowed portion 322 was simulated as being about 42% less the peripheral blood steal 1008 occurring within the subject 104 implanted with the non-restrictive arteriovenous graft 202.

According to at least one study, such as is found in Sutera, S. P. and Mehrjardi, M.H., Deformation and Fragmentation of Human Red Blood Cells in Turbulent Shear Flow, Biophysical Journal, Vol. 5 (1975): 1-10, wall shear stress 1010 in an arteriovenous graft 202 or graft system 200 should be less than approximately 2000 dynes/centimeter². As shown in the summary chart 1000, the wall shear stress 1010 is 135 dynes/centimeter² and 400 dynes/centimeter² in the non-restrictive arteriovenous graft 202 and the restrictive arteriovenous graft system 200, respectively.

Using information about the wall shear stress 1010, platelet stimulation factor 1012 and predicted percent hemolysis 1014 can be calculated. The platelet stimulation factor 1012 can be calculated by taking the product of (wall shear stress)×(blood residence time in the arteriovenous graft)^{0.452}. According to Wootton, D. M. and Ku, D. N., Fluid Mechanics of Vascular Systems, Diseases, and Thrombosis, Annu. Rev. Biomed. Eng. (1999) 01:299-329, the platelet stimulation factor 1012 should be less than 1000. As shown in the summary chart 1000, the platelet stimulation factor 1012 is 200 and 650 in the non-restrictive arteriovenous graft 202 and the restrictive arteriovenous graft system 200, respectively. The predicted percent hemolysis 1014 can be estimated using a model formula proposed by Giersiepen, M., Wurzinger, L. J., Opitz, R., and Reul, H., Estimation of Shear Stress-Related Blood Damage in Heart Valve Protheses—in vitro Comparison of 24 Aortic Valves, The International Journal of Artificial Organs 13.5 (1990): 300-306. According to Giersiepen et al., the predicted percent hemolysis 1014 is equal to the product of (3.62×10⁻⁵)×(wall shear stress (in Pa))^{2.416}×(blood residence time in the arteriovenous graft)^{0.785}. As shown in the summary chart 1000, predicted percent hemolysis is 2.2, 6.1, and 7.6 in the non-restrictive arteriovenous graft 202, the arteriovenous graft system 200 including the 25 millimeter long restrictor narrowed portion 322, and the arteriovenous graft system 200 including the 45 millimeter long restrictor narrowed portion 322, respectively.

Other simulated information summarized in the chart 1000 includes the maximum strain rate in the arteriovenous graft 1016 and the maximum strain rate at the graft-artery anastomosis 1018. As shown, the maximum strain rate in graft 1016 is simulated as being 2000 and 18000 in the non-restrictive arteriovenous graft 202 and the restrictive arteriovenous graft system 200, respectively; while the maximum strain rate at the graft-artery anastomosis 1018 is simulated as being 20000 and 10000, respectively.

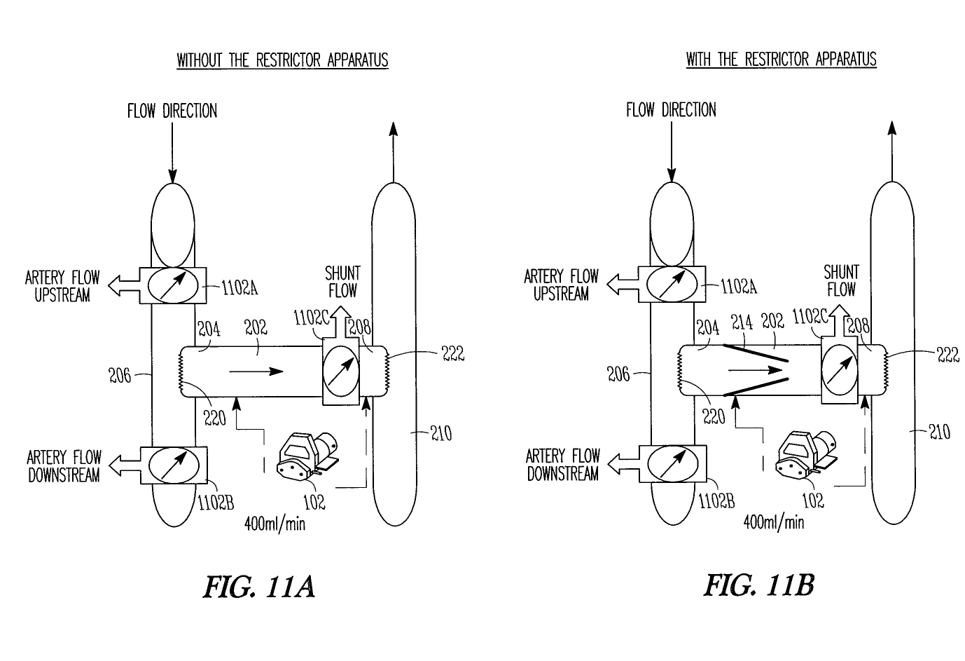

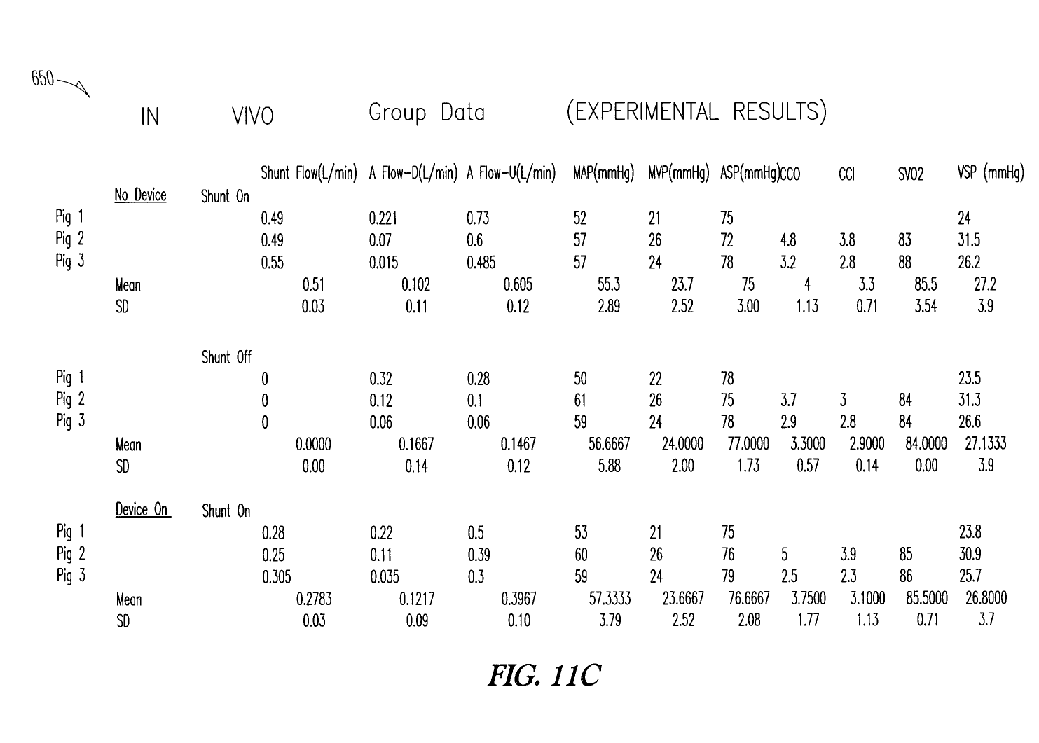

To experimentally illustrate the utility of the present blood flow restrictor apparatus 214, in vivo experiments were performed on three pigs ranging in body weight from about 44.0-47.7 kilograms. In each of the pigs, as respectively shown in FIGS. 11A and 11B, an arteriovenous graft 202 or an arteriovenous graft system 202 (including an arteriovenous graft 202 and a restrictor apparatus 214) was subcutaneously implanted. Each arteriovenous graft 202 extended from an arterial end portion 204 to a venous end portion 208. The arterial end portion 204 was anastomosed 220 to a pig's artery (e.g., iliac artery) 206, while the venous end portion 208 was anastomosed 222 to a pig's vein (e.g., iliac vein) 210.

Each of the pigs was further instrumented with one or more measurement devices, such as one or more blood flow rate detectors 1102A-C, blood pressure detectors, SVO2 detectors, or respiration detectors, for data gathering purposes. Some of the parameters measured by the one or more measurement devices included iliac blood flow upstream to the arteriovenous graft 202, iliac blood flow downstream to the arteriovenous graft 202, blood flow through the arteriovenous graft 202, mean aortic blood pressure, systolic blood pressure, mean iliac venous pressure upstream of the arteriovenous graft 202, continuous cardiac output, continuous cardiac index, and SVO2. FIGS. 11A and 11B illustrate example placement of three blood flow rate detectors 1102A-C used to measure iliac blood flow upstream to the arteriovenous graft 202, iliac blood flow downstream to the arteriovenous graft 202, and blood flow through the arteriovenous graft 202. As shown, a first blood flow rate detector 1102A can be disposed upstream of the arteriovenous graft 202 in the iliac artery 206, a second blood flow rate detector 1102B can be disposed downstream of arteriovenous graft 202 in the iliac artery 206, and a third blood flow rate detector 1102C can be disposed in the arteriovenous graft 202.

Using the three blood flow rate detectors 1102A-C, blood flow rates through each pig were measured with (FIG. 11B) and without (FIG. 11A) the restrictor apparatus 214. In addition, blood flow rates through each pig were measured with and without a dialysis machine 102 present. As discussed above, blood from each pig can be drawn via an arterial cannula 108 (FIG. 2C) at the arterial side 204 of the arteriovenous graft 202 and received by the dialysis machine 102 where it is dialyzed. After being dialysized, the blood can be returned to the pg at the venous side 208 of the arteriovenous graft 202 via a venous cannula 110 (FIG. 2C). For this in vivo experiment, blood was drawn from the arteriovenous graft 202, via the arterial cannula 108, at a rate of 400 milliliters per minute.

FIGS. 11C-11E provide a data chart 1150 summarizing the results of the in vivo experimentation performed on the three pigs. In brief, when the dialysis machine 102 was turned off, it was found that on average blood flow via the arteriovenous graft 202 was reduced (0.51+/−0.03 vs. 0.28+/−0.03 liters/minute) when the restrictor apparatus 214 was present (i.e., integrated with the arteriovenous graft 202 as a unitary body or interposed between the arterial 204 and venous 208 end portions of the arteriovenous graft 202). Without the restrictor apparatus 214 present, the arteriovenous graft 202 on average caused iliac blood flow to increase from 0.15+/−0.12 to 0.61+/−0.12 liters/minute (306.7%). With the restrictor apparatus 214 present, the arteriovenous graft 202 on average caused iliac blood to increase from 0.15+/−0.12 to 0.40+/−0.1 liters/minute (166.7%).

Other information gleaned from the in vivo experimentation performed on the three pigs is as follows. It was found that sufficient blood flow for performing hemodialysis can still be obtained acutely after implanting the restrictor apparatus 214 in the arteriovenous graft 202. Regarding CO (which was measured in two of the three pigs), it was found that the arteriovenous graft 202 caused CO to increase from 3.7 to 4.8 liters/minute (29.7%) and from 2.9 to 3.2 liters/minute (9.4%)—an average increase of 21%—without the restrictor apparatus 214 present. With the restrictor apparatus 214 present, the arteriovenous graft 202 caused CO to increase from 4.1 to 5 liters/minute (22%) and from 2.1 to 2.5 liters/minute (19.1%)—an average increase of also 21%. It was further found that arterial pressure, systolic aortic pressure, and mean iliac venous pressure were not substantially altered depending on whether or not the restrictor apparatus 214 was or was not present.

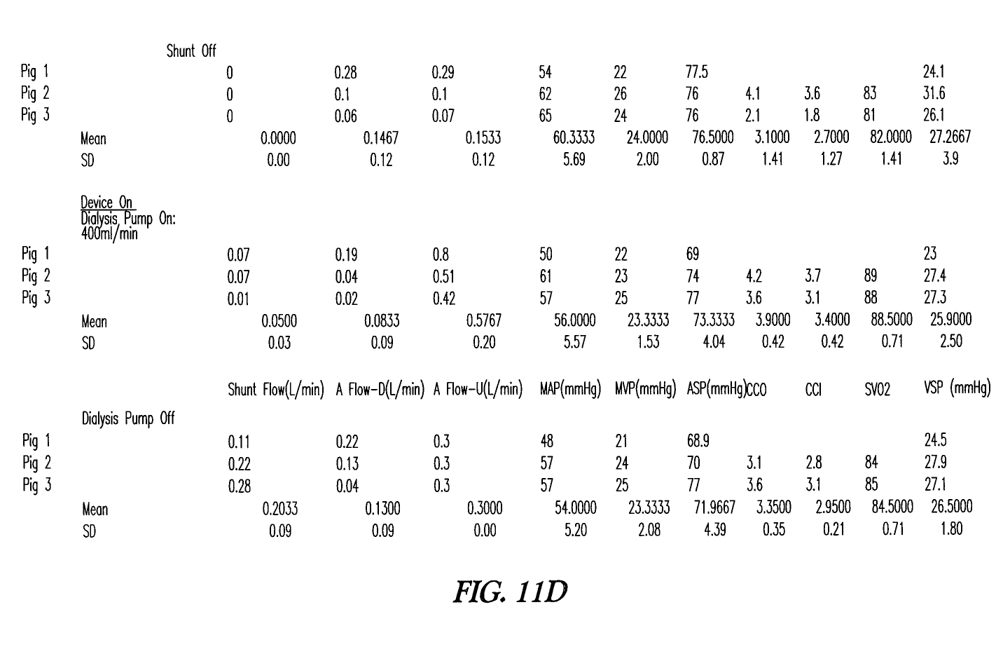

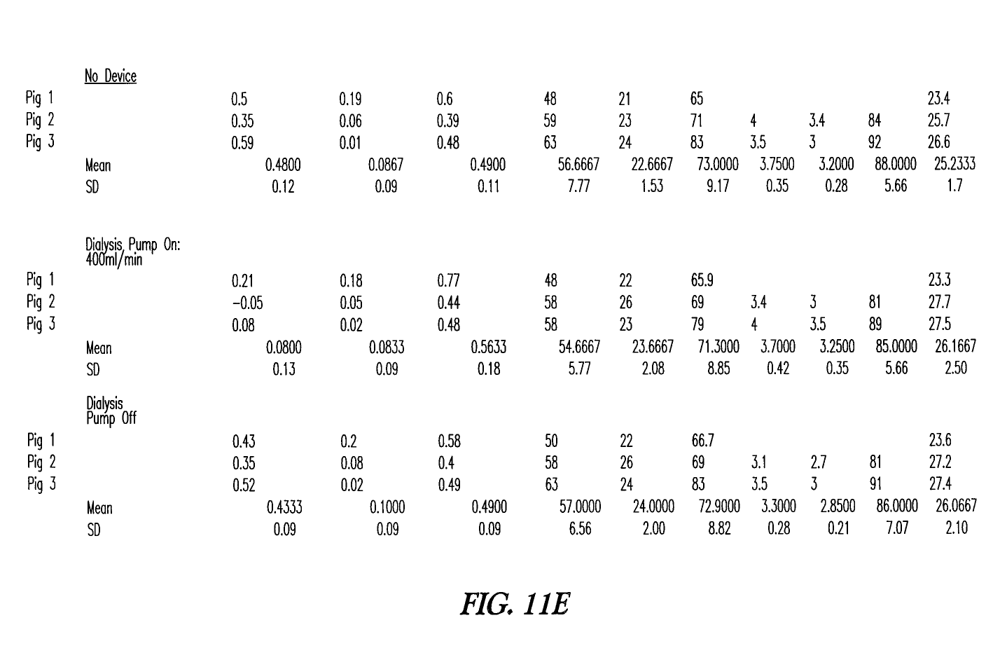

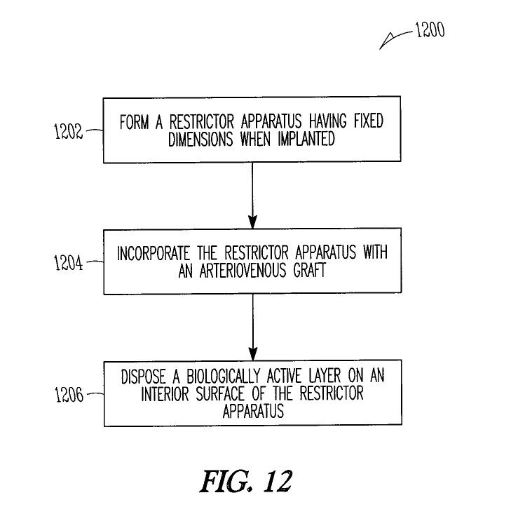

FIG. 12 illustrates an example method 1200 of forming an arteriovenous graft system. At 1202, a restrictor apparatus having fixed dimensions, when implanted, is formed. According to varying examples, forming the restrictor apparatus comprises forming a restrictor entry portion, a restrictor exit portion, and a optionally a restrictor narrowed portion therebetween. The restrictor entry portion includes a convergent first lumen that tapers outward on a first end to substantially match an interior diameter of an arterial portion of an arteriovenous graft. In one example, the first lumen includes an entry angle of less than or equal to about 6 degrees between the wall of the first lumen and a coaxial axis defining a center of the first lumen. In another example, the first lumen includes a convergent curved wall having a radius of curvature of at least 2 millimeters.

Options for the restrictor exit and restrictor narrowed portions are as follows. In varying examples, the restrictor exit portion includes a divergent second lumen that tapers outward on a second end to substantially match an interior diameter of a venous portion of the arteriovenous graft. In one example, the second lumen includes an exit angle of less than or equal to about 6 degrees between the wall of the second lumen and a coaxial axis defining a center of the second lumen. In varying examples, the restrictor narrowed portion includes a third lumen connecting the first and second lumens. The third lumen is narrower than at least a portion of the first and second lumens and substantially matches adjacent interior diameters of the first and second lumens (i.e., substantially matches a second end of the first lumen and a first end of the second lumen). In one example, the third lumen includes an effective interior diameter of at least 2.5 millimeters.

At 1204, the restrictor apparatus is incorporated with an arteriovenous graft. According to certain examples, the incorporation of the restrictor apparatus with the arteriovenous graft includes cutting the arteriovenous graft between an arterial and a venous end portion thereof and securely coupling the restrictor apparatus to such graft portions. According to other examples, the incorporation of the restrictor apparatus with the arteriovenous graft includes disposing the restrictor apparatus within an interior diameter wall of the arteriovenous graft. According to still other examples, the incorporation of the restrictor apparatus with the arteriovenous graft includes the formation of an arteriovenous graft having an integrated restrictor apparatus. Optionally, at 1206, an interior surface of at least one of the first, second, or third lumens of the restrictor apparatus is coated with a biologically active layer.

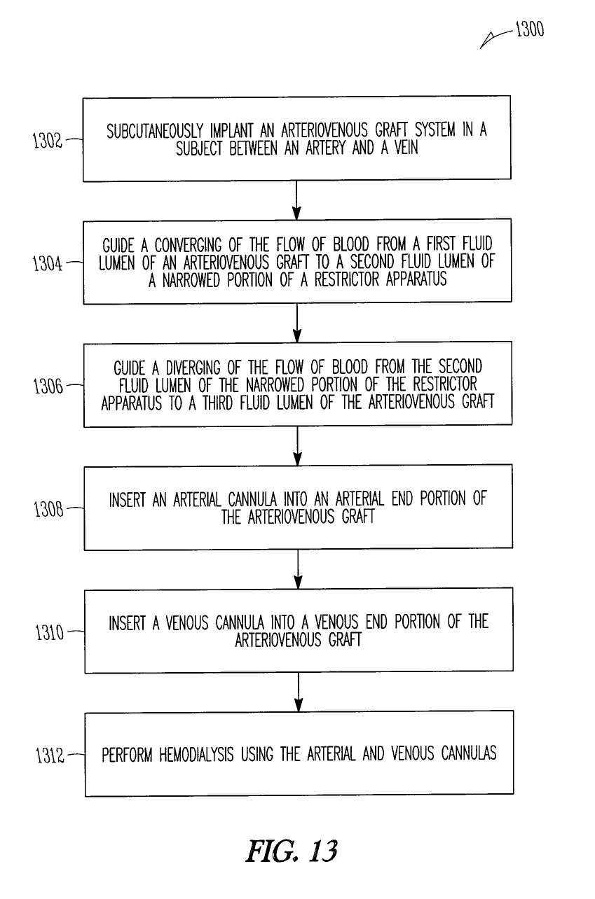

FIG. 13 illustrates an example method 1300 of restricting a flow of blood through an arteriovenous graft system including an arteriovenous graft and at least one restrictor apparatus. At 1302, the arteriovenous graft system is subcutaneously implanted within a subject between a subject's artery and vein. The at least one restrictor apparatus is located between an arterial end portion and a venous end portion of the arteriovenous graft. The arterial end portion of the arteriovenous graft is anastomosed to the subject's artery, while the venous end portion of the arteriovenous graft is anastomosed to the subject's vein.

At 1304, a converging of a flow of blood from a first fluid lumen defined by a first interior diameter wall of the arteriovenous graft is guided to a second fluid lumen defined by a fixed interior diameter wall of a narrowed portion of the at least one restrictor apparatus. At 1306, a diverging of the flow of blood from the second fluid lumen defined by the fixed interior diameter wall of the narrowed portion of the restrictor apparatus is guided to a third fluid lumen defined by a second interior diameter wall of the arteriovenous graft.

At 1308, an arterial cannula is inserted into the arterial end portion of the arteriovenous graft, and at 1310, a venous cannula is inserted into the venous end portion of the arteriovenous graft. At 1312, hemodialysis is performed on the flow of blood drawn by the arterial cannula and thereafter, the cleansed blood returned to the subject via the venous cannula. During the hemodialysis, the blood flow bypassing the arterial and venous cannulas through the arteriovenous graft is restricted using the restrictor apparatus. Upon completion of the hemodialysis, the arterial and venous cannulas are removed from the respective arterial and venous end portions of the arteriovenous graft.

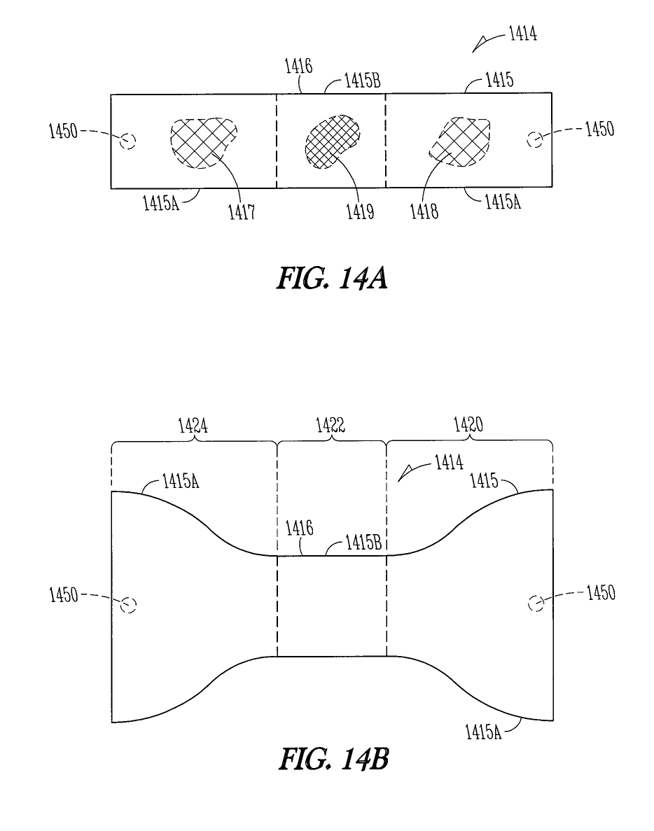

FIGS. 14A and 14B illustrate an example of a restrictor apparatus 1414 configured to be placed within a destination vessel for an arteriovenous hemodialysis access, such as an arteriovenous graft or an arteriovenous fistula. In some examples, the restrictor apparatus 1414 can be endovascularly inserted into the arteriovenous hemodialysis access. In some examples, the restrictor apparatus 1414 includes a tubular portion 1415 including a delivery configuration (FIG. 14A) and a deployed configuration (FIG. 14B). In further examples, the restrictor apparatus 1414 includes a size-limiting portion 1416 configured to constrain a size of the tubular portion 1415 in the deployed configuration. In an example, the size-limiting portion 1416 is proximate a middle of the restrictor apparatus 1414, although it is contemplated that the size-limiting portion 1416 be positioned at any point or points along the restrictor apparatus 1414 that size-limiting of the restrictor apparatus 1414 would be desirable. In an example, the size-limiting portion 1416 is integrally formed with the tubular portion 1415. In an example, the tubular portion 1415 includes a first portion 1415A including a first size substantially matching an interior size of the destination vessel with the tubular portion 1415 in the deployed configuration. In an example, the tubular portion 1415 includes a second portion 1415B constrained by the size-limiting portion 1416 to include a second size smaller than the first size of the tubular portion 1415. In an example, the second portion 1415B of the tubular portion 1415 is integrally formed with the size-limiting portion 1416. The destination vessel of these examples differs depending upon the type of arteriovenous hemodialysis access. For instance, the destination vessel of an arteriovenous graft can be the graft itself, and the destination vessel of an arteriovenous fistula can be one of the surgically connected vessels of the fistula.

In some examples, the restrictor apparatus 1414 includes a stent-graft construction including a stent-like apparatus covered with a substantially impermeable coating, membrane, or other material to form a wall of the restrictor apparatus 1414 having a lumen therethrough. This wall of the restrictor apparatus 1414, in some examples, acts to inhibit or prevent blood or other fluids from passing through the wall of the restrictor apparatus 1414. In some examples, the stent-like apparatus can be formed from one or more materials including, but not limited to, a shape memory metal, such as Nitinol. In other examples, the stent-like apparatus can be formed from one or more materials including, but not limited to, stainless steel, cobalt chromium, or any deformable alloy. In some examples, the substantially impermeable coating, membrane, or other material can include, for instance, Dacron or polytetrafluoroethylene (PTFE). The substantially impermeable material, in some examples, can stretch when the restrictor apparatus 1414 is expanded, as described herein, to define a substantially fluid impermeable wall to perform blood flow restriction, as described herein.

In an example, the stent-like apparatus includes a pattern cut into the stent-like apparatus, which can include a mesh-like or weave-like pattern, although in other examples, other patterns are contemplated. The pattern, in some examples, allows the restrictor apparatus 1414 to expand once deployed. By varying the pattern of the restrictor apparatus 1414, different expansion characteristics can be obtained. For instance, as shown in the cut-away portions of FIG. 14A, the tubular portion 1415 includes a first pattern 1417 at a first end and a second pattern 1418 at a second end. In an example, the first and second patterns 1417, 1418 are substantially similar so that the first and second patterns 1417, 1418 expand to a similar size to one another. However, in other examples, the first and second patterns 1417, 1418 can differ from one another to allow for different expansion characteristics of the first and second ends when deployed.

In an example, the size-limiting portion 1416 of the restrictor apparatus 1414 includes a third pattern 1419 which differs from the first and second patterns 1417, 1418. In an example, the third pattern 1419 is configured to inhibit expansion of the restrictor apparatus 1414 more than the first and second patterns 1417, 1418. In an example, the restrictor apparatus 1414, when deployed, includes a smaller section, constrained by the size-limiting portion 1416, and larger portions, as determined by the first and second patterns 1417, 1418. In a further example, the size limiting portion 1416 is included substantially at the center of the restrictor apparatus 1414 to maintain a relatively small size of the center, when the restrictor apparatus 1414 is deployed, and allow for the ends, with the first and second patterns 1417, 1418, to flare outwardly from the size-limiting portion 1416 to the respective ends, which, in an example, are constrained by the inner geometry of the destination vessel. In this example, the deployed restrictor apparatus 1414 resembles an hourglass in shape. However, in other examples, other shapes are contemplated, depending upon the desired blow flow restriction characteristics.

In some examples, the first, second, and third patterns 1417, 1418, 1419 of the restrictor apparatus 1414 are formed by cutting away material from a tube. In an example, the material can be cut away using a laser cutter. In various examples, the first, second, and third patterns 1417, 1418, 1419 are configured to expand when the restrictor apparatus 1414 is deployed. In the example shown in FIG. 14A, the first, second, and third patterns 1417, 1418, 1419 each include mesh-like patterns. However, in further examples, other patterns are contemplated, such as zig-zag patterns, wavy patterns, spiral patterns, weave patterns, or the like. In other examples, the first, second, and third patterns 1417, 1418, 1419 can include a combination of patterns. In further examples, the first, second, and third patterns 1417, 1418, 1419 need not all be similar patterns. In another example, one or more sections can remain uncut. For instance, the size-limiting portion 1416 of the restrictor apparatus 1414 can remain uncut, such that the third pattern 1419 can be that of an uncut tube (rather than the mesh-like pattern shown in FIG. 14A), and the end portions of the restrictor apparatus 1414 include the first and second patterns 1417, 1418 configured to allow expansion of the end portions of the restrictor apparatus 1414. In this way, when deployed, the restrictor apparatus 1414 fans out at the end portions, but the uncut size-limiting portion 1416 remains at the same size and does not expand during deployment.

In other examples, further techniques can be used to obtain different expansion characteristics of the restrictor apparatus 1414. For instance, in an example, differential molding can be performed at the time the restrictor apparatus 1414 is manufactured to achieve of the restrictor apparatus 1414. In some examples, the restrictor apparatus 1414 includes a first molded portion 1417 and a second molded portion 1419, wherein the first molded portion 1417 includes a different expansion characteristic from the second molded portion 1419. In an example, the first molded portion 1417 can be configured to constrain a first portion 1420 and/or 1424 of the tubular portion to the first size, and a second molded portion 1419 can be configured to constrain a second portion 1422 of the tubular portion 1416 to the second size. In further examples, a combination of techniques can be used to obtain different expansion characteristics of the restrictor apparatus 1414.

In an example, the stent-like apparatus of the restrictor apparatus 1414 is formed from a shape memory metal, such as Nitinol. The “shape memory” property permits the restrictor apparatus 1414 to “remember” a previous shape. For example, the shape memory restrictor apparatus 1414 can be compressed or otherwise deformed (e.g., compressed within a sleeve), and can then return toward or regain its pre-deformation shape when uncompressed or otherwise released (e.g., when the sleeve is removed). In other examples, the stent-like apparatus of the restrictor apparatus 1414 is formed from one or more materials including, but not limited to, stainless steel, cobalt chromium, or any deformable alloy. If formed from such materials without shape memory properties, the restrictor apparatus 1414 can be deployed using a balloon system (as described herein) or other apparatus configured to facilitate expansion of the restrictor apparatus 1414.

In an example, the restrictor apparatus 1414 can include an entry portion 1420. The entry portion 1420 can include a convergent first lumen portion that tapers to substantially match an interior diameter of an arterial side of the destination vessel. In an example, the restrictor apparatus 1414 includes an exit portion 1424. The exit portion 1424 can include a divergent second lumen portion that tapers to substantially match an interior diameter of a venous side of the destination vessel. In an example, the restrictor apparatus 1414 can include an intermediate portion 1422 between the entry portion 1420 and the exit portion 1424. In an example, the intermediate portion 1422 can include a substantially cylindrical third lumen portion.

In some examples, the restrictor apparatus 1414 includes an imageable feature 1450 configured to be viewable in an imaging modality. In an example, the imageable feature 1450 includes a radio opaque feature for radiographic imaging, an ultrasound reflective feature for ultrasonic imaging, or a feature that allows imaging in a different modality. In a further example, the imageable feature 1450 includes a combination of features for imaging in two or more imaging modalities. Although shown as a circular feature, the imageable feature 1450 can include any shape or configuration. In an example, the imageable features 1450 are disposed proximate the ends of the restrictor apparatus 1414. This allows for imaging of the ends of the restrictor apparatus 1414, for instance, to facilitate a caregiver in identifying the ends of the restrictor apparatus 1414 and avoid puncturing the restrictor apparatus 1414 during a procedure, such as a dialysis procedure.

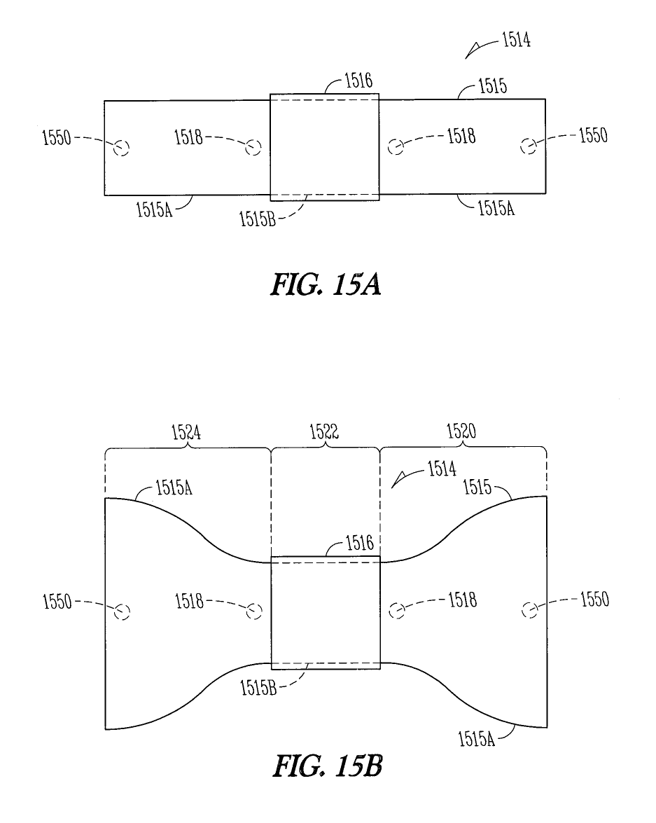

Referring to FIGS. 15A and 15B, there is shown an example of a restrictor apparatus 1514 configured to be placed within a destination vessel for an arteriovenous hemodialysis access, such as an arteriovenous graft or an arteriovenous fistula. In some examples, the restrictor apparatus 1514 can be endovascularly inserted into the arteriovenous hemodialysis access. In some examples, the restrictor apparatus 1514 includes a tubular portion 1515 including a delivery configuration (FIG. 15A) and a deployed configuration (FIG. 15B). In further examples, the restrictor apparatus 1514 includes a size-limiting portion 1516 configured to constrain a size of the tubular portion 1515 in the deployed configuration. In an example, the size-limiting portion 1516 is proximate a middle of the restrictor apparatus 1514, although it is contemplated that the size-limiting portion 1516 be positioned at any point or points along the restrictor apparatus 1514 that size-limiting of the restrictor apparatus 1514 would be desirable. In an example, the tubular portion 1515 includes a first portion 1515A including a first size substantially matching an interior size of the destination vessel with the tubular portion 1515 in the deployed configuration. In an example, the tubular portion 1515 includes a second portion 1515B constrained by the size-limiting portion 1516 to include a second size smaller than the first size of the tubular portion 1515. The destination vessel of these examples differs depending upon the type of arteriovenous hemodialysis access. For instance, the destination vessel of an arteriovenous graft can be the graft itself, and the destination vessel of an arteriovenous fistula can be one of the surgically connected vessels of the fistula.

In some examples, the restrictor apparatus 1514 includes a stent-graft construction including a stent-like apparatus covered with a substantially impermeable coating, membrane, or other material to form a wall of the restrictor apparatus 1514 having a lumen therethrough. This wall of the restrictor apparatus 1514, in some examples, acts to inhibit or prevent blood or other fluids from passing through the wall of the restrictor apparatus 1514. In some examples, the stent-like apparatus can be formed from one or more materials including, but not limited to, a shape memory metal, such as Nitinol. In other examples, the stent-like apparatus can be formed from one or more materials including, but not limited to, stainless steel, cobalt chromium, or any deformable alloy. In some examples, the substantially impermeable coating, membrane, or other material can include, for instance, Dacron or polytetrafluoroethylene (PTFE). The substantially impermeable material, in some examples, can stretch when the restrictor apparatus 1514 is expanded, as described herein, to define a substantially fluid impermeable wall to perform blood flow restriction, as described herein.

In some examples, the size-limiting portion 1516 includes a fixed ring 1516 disposed around the second portion 1515B of the tubular portion 1515 to limit the size of the second portion 1515B upon deployment of the restrictor apparatus 1514. In some examples, the fixed ring 1516 can be formed from cobalt chromium, stainless steel, or some other material. Although shown as a band having a certain thickness, the fixed ring 1516 can be any size or shape, provided it is capable of being implanted in the desired manner. In an example, the fixed ring 1516 can include a single wire rather than a band. For instance, the fixed ring 1516 of this example can be a single stent wire or the like. Also, in other examples, more than one fixed ring 1516 can be used with the restrictor apparatus 1514, depending upon the desired blood flow characteristics to be achieved. In various examples, the fixed ring 1516 can be placed at any point along the tubular portion 1515, and need not be placed proximate the center of the tubular portion 1515, as shown in FIGS. 15A and 15B. In these examples, the fixed ring 1516 includes a fixed or substantially fixed diameter, such that the fixed ring 1516 acts to constrain expansion of the stent-like apparatus of the tubular portion 1515 during deployment of the restrictor apparatus 1514, for instance, as shown in FIG. 15B. In various examples, the fixed ring 1516 has a diameter between 1 and 6 mm. In one example, the diameter of the fixed ring 1516 is 2 mm.

In other examples, the size-limiting portion 1516 can include a stent (similar to the moldable stent 918 discussed above) instead of the fixed ring 1516, as shown. In these examples, the stent can be configured to expand only a certain amount, which is less than the amount the tubular portion 1515 is configured to expand. In this way, when the restrictor apparatus 1514 is deployed, expansion of the second portion 1515B of the tubular portion 1515 is constrained by the stent and the first portions 1515A of the tubular portion 1515 are able to expand to the size of the destination vessel or otherwise to the predetermined expanded size of the tubular portion 1515. Such a configuration potentially allows for smaller insertion size than the fixed ring 1516 discussed above.

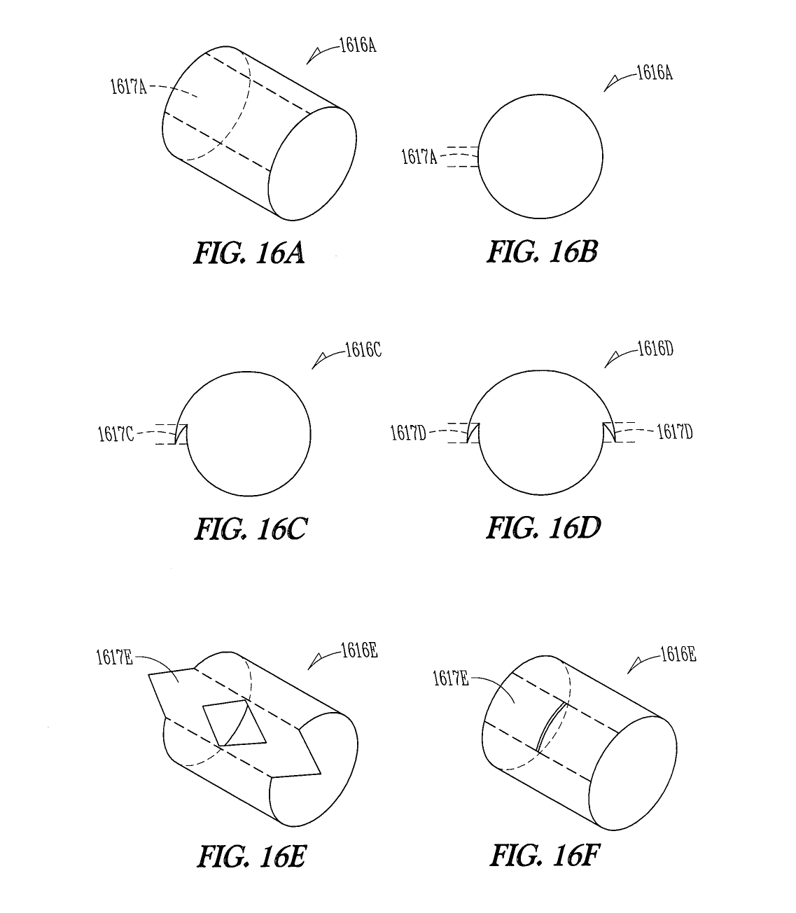

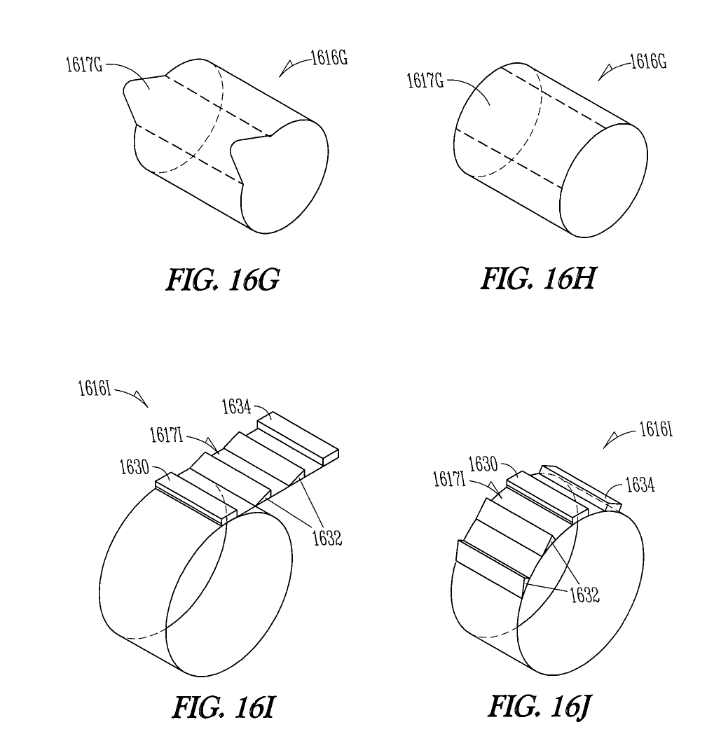

Referring to FIGS. 16A-16J, various other examples of size-limiting portions are shown, for instance, for use with the tubular portion 1515 of FIGS. 15A and 15B. Referring initially to FIGS. 16A and 16B, a size-limiting portion 1616A can be substantially similar to the fixed ring 1516, differing in that the size-limiting portion 1616A includes at least one expandable area 1617A disposed at at least one point along the size-limiting portion 1616A. Although FIGS. 16A and 16B show only one such expandable area 1617A, in other examples, two or more expandable areas 1617B can be included at points around the size-limiting portion 1616A. In some examples, the one or more expandable areas 1617A allow the size-limiting portion 1616A to expand during deployment of the restrictor apparatus 1514 to allow for smaller insertion size than the fixed ring 1516 discussed above, such that the size-limiting portion 1616A, when deployed, could include a diameter larger than that the size-limiting portion 1616A during delivery. In various examples, the size-limiting portion 1616A can expand with self-expansion of the restrictor apparatus, self-expansion of the size-limiting portion 1616A, deployment using a balloon catheter, or deployment in another way. Various examples of expandable areas are contemplated herein. Although some examples are described below, it should be understood that numerous other examples are contemplated. Such examples can also include combinations of two or more of the expandable area examples discussed herein or otherwise contemplated.

Referring to FIG. 16C, a size-limiting portion 1616C includes an expandable area 1617C that includes a folded, crimped, or otherwise bent, such that the size-limiting portion 1616C includes “extra” material. Such a configuration allows for a smaller diameter for delivery but also allows for the size-limiting portion 1616C to expand slightly during deployment of the restrictor apparatus with self-expansion of the restrictor apparatus, self-expansion of the size-limiting portion 1616C, deployment using a balloon catheter, or deployment in another way.

Referring to FIG. 16D, in an example, a size-limiting portion 1616D is substantially similar to the size-limiting portion 1616C except that it includes two expandable areas 1617D. In other examples, the size-limiting portion 1616D can include more than two expandable areas 1617D. The number and location of the one or more expandable areas of various examples can depend upon the desired amount of expansion and/or the desired blood flow characteristics to be obtained using the size-limiting portion 1616C, 1617D with the particular restrictor apparatus.

Referring to FIGS. 16E and 16F, in some examples, an expandable portion 1617E of a size-limiting portion 1616E can include a partial stent-like configuration. In this example, the size-limiting portion 1616E can include one or more stent-like expandable portions 1617E (only one is shown in FIG. 16E). The one or more expandable portions 1617E can include one or more “cells” cut into the size-limiting portion 1616E, the one or more “cells” configured to allow the size-limiting portion 1616E to expand from a delivery configuration (FIG. 16E) to a deployed configuration (FIG. 16F) with self-expansion of the restrictor apparatus, self-expansion of the size-limiting portion 1616E, deployment using a balloon catheter, or deployment in another way.

In an example, the expandable portion 1617E is formed by cutting a stent-like pattern in a portion of the size-limiting portion 1616E and leaving the remainder of the size-limiting portion 1616E uncut. In this example, the expandable portion 1617E is integral with the remainder of the size-limiting portion 1616E. In an example, such a stent-like pattern can be cut in the size-limiting portion 1616E using laser cutting or some other precision cutting means. In an example, the size of the expandable portion 1617E can vary according to the amount of available expansion desired for the size-limiting portion 1616E. For instance, a size-limiting portion 1616E including an expandable portion 1617E forming 30% of the circumference of the size-limiting portion 1616E includes less available expansion than a size-limiting portion 1616E including a similar expandable portion 1617E forming 40% of the circumference of the size-limiting portion 1616E. In various examples, the expandable portion 1617E can form any percentage of the circumference of the size-limiting portion 1616E, depending upon the desired amount of available expansion in the size-limiting portion 1616E. In further examples, the expandable portion 1617E can include various stent-like patterns cut into the expandable portion 1617E, depending upon the desired amount of expansion for the size-limiting portion 1616E.

Referring to FIGS. 16G and 1611, in some examples, an expandable portion 1617G of a size-limiting portion 1616G can include one or more wave-like and/or zig-zag portions (only one is shown in FIG. 16G). The one or more expandable portions 1617G can include one or more wave-like or zig-zag portions cut into the size-limiting portion 1616G, the one or more wave-like or zig-zag portions configured to allow the size-limiting portion 1616G to expand from a delivery configuration (FIG. 16G) to a deployed configuration (FIG. 16H) with self-expansion of the restrictor apparatus, self-expansion of the size-limiting portion 1616G, deployment using a balloon catheter, or deployment in another way.

Referring to FIGS. 16I and 16J, in some examples, an expandable portion 1617I of a size-limiting portion 1616I can include one or more expandable strap portions (only one is shown in FIGS. 16I and 16J). In an example, the one or more expandable strap portions 1617I are configured in a manner similar to a cable tie or zip tie, except that the arrangement is reversed. That is, instead of locking as the size-limiting portion 1616I contracts, the size-limiting strap portion 1616! locks as it expands. In some examples, the expandable strap portion 16171 includes a locking member 1630 configured to allow one or more locking teeth 1632 to pass through in an expanding direction of the size-limiting portion 1616I but not in a contracting direction of the size-limiting portion 1616I. In an example, the expandable strap portion 1617I can include a stop 1634 configured to stop expansion of the expandable strap portion 1617I, to inhibit the size-limiting portion 1616I from over-expanding and/or uncoupling to cease forming a ring-like size-limiting portion 1616I. In various examples, this configuration of the size-limiting portion 1616I allows the size-limiting portion 16161 to have a lower profile in, for instance, a delivery configuration (FIG. 16I) and to expand to, for instance, a deployed configuration (FIG. 16J) with self-expansion of the restrictor apparatus, deployment using a balloon catheter, or deployment in another way.

In some examples, restrictor apparatuses can include one or more of the size-limiting portions of FIGS. 16A-16J, as described herein. In various examples, it is contemplated that such restrictor apparatuses including one or more of the size-limiting portions of FIGS. 16A-16J can be deployed in various ways, as described herein, including self-expansion of the restrictor apparatus, deployment using a balloon catheter, or deployment in another way. For examples including deployment using a balloon catheter, a physician, operator, or other user deploying the restrictor apparatus can determine the final size of the size-limiting portion by inflating the balloon of the balloon catheter an amount sufficient to achieve the desired deployed diameter of the size-limiting portion. In some examples, the size-limiting portion can be left at its delivery configuration size (not expanded), expanded completely, or expanded to a dimension between the delivery configuration size and complete expansion. That is, the final delivery dimension of the size-limiting portion can be a function of the amount that the balloon of the balloon catheter is inflated. For instance, the final dimension of the size-limiting portion can be determined and the physician, operator, or other user deploying the restrictor apparatus can inflate the balloon an amount sufficient to achieve this desired final dimension of the size-limiting portion.

Referring again to FIGS. 15A and 15B, in some examples, the restrictor apparatus 1514 can include various manners of maintaining the size-limiting portion 1516 (including any of the example size-limiting portions shown in FIGS. 16A-16I or otherwise contemplated herein) at the desired location along the tubular portion 1515. In an example, the restrictor apparatus 1514 can include one or more retainers 1518 disposed on either side of the size-limiting portion 1516 to maintain the size-limiting portion 1516 in place. In various examples, the retainers 1518 of the restrictor apparatus 1514 can be welded, adhesively attached, or otherwise affixed to the tubular portion 1515. In some examples, the retainers 1518 are integrally formed with the tubular portion 1515. In some examples, the retainers 1518 can be configured in shapes different from those shown in FIGS. 15A and 15B. For instance, in various examples, the retainers 1518 can include pins, elongate protrusions, bumps, ribs (extending partially or completely around the perimeter of the tubular portion 1515), or the like.

In other examples, the size-limiting portion 1516 can be maintained in place on the restrictor apparatus 1514 using frictional engagement. For instance, the size-limiting portion 1516 can include an unpolished or otherwise roughened inner surface (either partial surface or the entire inner surface) which is configured to increase friction between the size-limiting portion 1516 and the restrictor apparatus 1514 to aid in maintaining the size-limiting portion 1516 in place on the restrictor apparatus 1514.

In still other examples, the size-limiting portion 1516 can be maintained in place on the restrictor apparatus 1514 using magnetic engagement. For instance, the size-limiting portion 1516 can include a magnetized inner surface (either partial surface or the entire inner surface) which is configured to magnetically couple the size-limiting portion 1516 and the restrictor apparatus 1514 to aid in maintaining the size-limiting portion 1516 in place on the restrictor apparatus 1514.

In further examples, other manners of maintaining the size-limiting portion 1516 in place on the restrictor apparatus 1514 are contemplated. In still further examples, combinations of two or more manners of maintaining the size-limiting portion 1516 in place on the restrictor apparatus 1514 are contemplated.

In an example, the stent-like apparatus of the restrictor apparatus 1514 is formed from a shape memory metal, such as Nitinol. The “shape memory” property permits the restrictor apparatus 1514 to “remember” a previous shape. For example, the shape memory restrictor apparatus 1514 can be compressed or otherwise deformed (e.g., compressed within a sleeve), and can then return toward or regain its pre-deformation shape when uncompressed or otherwise released (e.g., when the sleeve is removed). In other examples, the stent-like apparatus of the restrictor apparatus 1514 is formed from one or more materials including, but not limited to, stainless steel, cobalt chromium, or any deformable alloy. If formed from such materials without shape memory properties, the restrictor apparatus 1514 can be deployed using a balloon system (as described herein) or other apparatus configured to facilitate expansion of the restrictor apparatus 1514.

In an example, the restrictor apparatus 1514 can include an entry portion 1520. The entry portion 1520 can include a convergent first lumen portion that tapers to substantially match an interior diameter of an arterial side of the destination vessel. In an example, the restrictor apparatus 1514 includes an exit portion 1524. The exit portion 1524 can include a divergent second lumen portion that tapers to substantially match an interior diameter of a venous side of the destination vessel. In an example, the restrictor apparatus 1514 can include an intermediate portion 1522 between the entry portion 1520 and the exit portion 1524. In an example, the intermediate portion 1522 can include a substantially cylindrical third lumen portion.

In some examples, the restrictor apparatus 1514 includes an imageable feature 1550 configured to be viewable in an imaging modality. In an example, the imageable feature 1550 includes a radio opaque feature for radiographic imaging, an ultrasound reflective feature for ultrasonic imaging, or a feature that allows imaging in a different modality. In a further example, the imageable feature 1550 includes a combination of features for imaging in two or more imaging modalities. Although shown as a circular feature, the imageable feature 1550 can include any shape or configuration. In an example, the imageable features 1550 are disposed proximate the ends of the restrictor apparatus 1514. This allows for imaging of the ends of the restrictor apparatus 1514, for instance, to facilitate a caregiver in identifying the ends of the restrictor apparatus 1514 and avoid puncturing the restrictor apparatus 1514 during a procedure, such as a dialysis procedure.

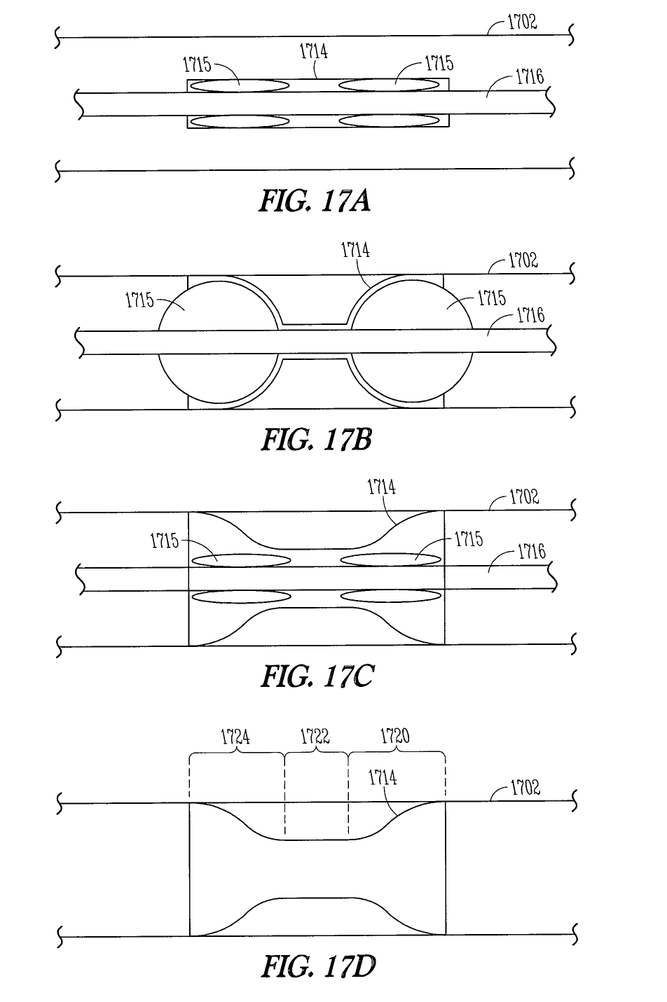

Referring to FIGS. 17A-17D, an example is shown of insertion of an example of a restrictor apparatus 1714, in the form of a moldable stent, into a destination vessel 1702 of an arteriovenous hemodialysis access. In various examples, it is contemplated that the restrictor apparatus 1714 can be used with any arteriovenous hemodialysis access, including an arteriovenous fistula, an arteriovenous graft, or the like. In further examples, insertion of restrictor apparatuses, such as the example restrictor apparatuses 1414, 1514 or any other restrictor apparatuses described herein, can be accomplished as described below.

In an example, a pair of deflated balloons 1715 and the restrictor apparatus 1714 can be endovascularly inserted within a destination vessel of an arteriovenous hemodialysis access, for instance, using a delivery catheter 1716 or a similar delivery technique. In some examples, the restrictor apparatus 1714 can be formed from a moldable material, such as metal. In an example, the restrictor apparatus 1714 can be formed from cobalt chromium, stainless steel, and/or another material. In an example, the restrictor apparatus 1714 can include a layer of a substantially impermeable material, such as, but not limited to, Dacron or PTFE. The substantially impermeable material layer, in some examples, can stretch when the restrictor apparatus 1714 is expanded, as described below, to define a substantially fluid impermeable wall to perform blood flow restriction, as described herein.

Once positioned at a desired location, for instance, within the destination vessel 1702 (see FIG. 17A), the balloons 1715 can be inflated within the restrictor apparatus 1714. In an example, the balloons 1715 can include a gap therebetween (see, e.g., FIG. 17B). The gap, in an example, can be positioned to coincide with a narrowed portion of the restrictor apparatus 1714 (or the size-limiting portion 1416, 1516 of the previously-described examples). In this way, the balloons 1715 can expand portions of the restrictor apparatus 1714 (in this example, end portions of the restrictor apparatus 1714) but expand the narrowed portion of the restrictor apparatus 1714 to a lesser extent or not at all.

In an example, inflating the balloons 1715 within the restrictor apparatus 1714 forces the restrictor apparatus 1714 outward to take a shape similar to that of the inflated balloons 1715. In an example, the restrictor apparatus 1714 can be expanded into engagement with an interior surface of the destination vessel 1702, such as to frictionally engage the restrictor apparatus 1714 with the destination vessel 1702.

In an example, once the restrictor apparatus 1714 is positioned and shaped, the balloons 1715 can be deflated (see FIG. 17C) and removed from within the restrictor apparatus 1714 (see FIG. 17D). The restrictor apparatus 1714 can maintain its shape similar to that of the inflated balloons 1715. In an example, the restrictor apparatus 1714 can be shaped to form an entry portion 1720 that can include a convergent first lumen that tapers to substantially match an interior diameter of an arterial portion of the destination vessel 1702. In an example, the restrictor apparatus 1714 can be shaped to form an exit portion 1724 that includes a divergent second lumen that tapers to substantially match an interior diameter of a venous portion of the destination vessel 1702. In another example, the restrictor apparatus 1714 can be shaped to form an intermediate portion 1722 between the entry portion 1720 and the exit portion 1724. In an example, the intermediate portion 1722 can include a substantially cylindrical third lumen. When shaped, in an example, the restrictor apparatus 1714 can form a generally continuous tubular wall defining a lumen therethrough. When inserted within the destination vessel 1702, in an example, the restrictor apparatus 1714, formed such as described above, can provide a restrictor apparatus to function in a manner similar to examples of restrictor apparatuses described herein.

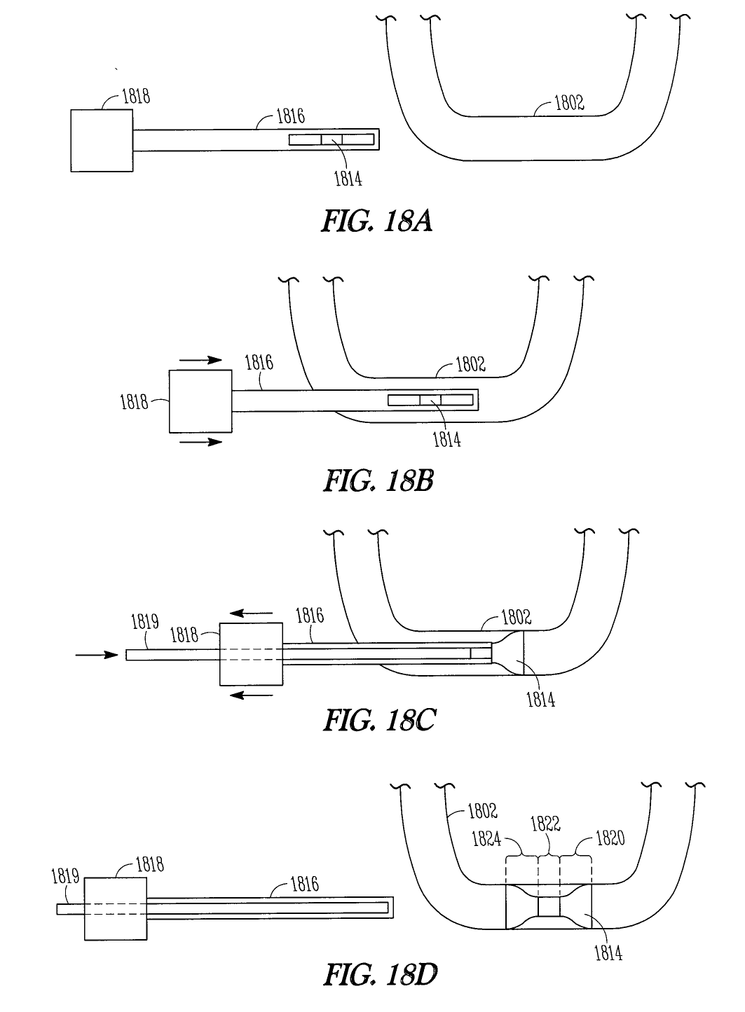

Referring to FIGS. 18A-18D, an example is shown of insertion of an example of a restrictor apparatus 1814, in the form of a moldable stent, into a destination vessel 1802 of an arteriovenous hemodialysis access. In various examples, it is contemplated that the restrictor apparatus 1814 can be used with any arteriovenous hemodialysis access, including an arteriovenous fistula, an arteriovenous graft, or the like. In further examples, insertion of restrictor apparatuses, such as the example restrictor apparatuses 1414, 1514 or any other restrictor apparatuses described herein, can be accomplished as described below.

In an example, the restrictor apparatus 1814 can be inserted within a destination vessel of an arteriovenous hemodialysis access, for instance, using a vascular sheath 1816. Such a delivery technique can be used, in some examples, as an alternative to catheter delivery or other delivery methods. In some examples, the restrictor apparatus 1814 can be formed from a shape memory material, such as Nitinol. In some examples, the restrictor apparatus 1814 can be formed from a moldable material, such as cobalt chromium, stainless steel, or another material. In an example, the restrictor apparatus 1814 can include a layer of a substantially impermeable material, such as, but not limited to, Dacron or PTFE. The substantially impermeable material layer, in some examples, can stretch when the restrictor apparatus 1814 is expanded, as described below, to define a substantially fluid impermeable wall to perform blood flow restriction, as described herein. In an example, the restrictor apparatus 1814 is placed within the vascular sheath 1816 (see FIG. 18A), and, if necessary, a deployment apparatus for the restrictor apparatus 1814, such as, but not limited to the example balloon deployment apparatuses described above.

The vascular sheath 1816, in some examples, is inserted within the destination vessel 1802 to position the restrictor apparatus 1814 at a desired location (see FIG. 18B). In some examples, the vascular sheath 1816 is placed into position using a delivery wire. In some examples, an obturator 1819 can be placed through a bleedback valve 1818 of the vascular sheath 1816 and used to push the restrictor apparatus 1814 out of the end of the vascular sheath 1816 (see FIG. 18C). In some examples the vascular sheath 1816 is withdrawn while pushing the restrictor apparatus 1814 out of the end of the vascular sheath 1816. The restrictor apparatus 1814, if formed from a shape memory material, can self-expand, as shown in FIG. 18C, as it is pushed from within the vascular sheath 1816, or can be expanded once fully pushed from within the vascular sheath 1816 using a balloon deployment apparatus or other such deployment apparatus. Once the restrictor apparatus 1814 is positioned and deployed, the vascular sheath 1816 and obturator 1819 can be fully retracted from within the destination vessel 1802 (see FIG. 18D).

In an example, the restrictor apparatus 1814 can be shaped to form an entry portion 1820 that can include a convergent first lumen that tapers to substantially match an interior diameter of an arterial portion of the destination vessel 1802. In an example, the restrictor apparatus 1814 can be shaped to form an exit portion 1824 that includes a divergent second lumen that tapers to substantially match an interior diameter of a venous portion of the destination vessel 1802. In another example, the restrictor apparatus 1814 can be shaped to form an intermediate portion 1822 between the entry portion 1820 and the exit portion 1824. In an example, the intermediate portion 1822 can include a substantially cylindrical third lumen. When shaped, in an example, the restrictor apparatus 1814 can form a generally continuous tubular wall defining a lumen therethrough. When inserted within the destination vessel 1802, in an example, the restrictor apparatus 1814, formed such as described above, can provide a restrictor apparatus to function in a manner similar to examples of restrictor apparatuses described herein.

In some examples, one or more of the restrictor apparatuses described above are configured to be tolerant to being crushed. That is, the one or more restrictor apparatuses are configured to resist being crushed or return to (or substantially return to) a pre-crush configuration (shape, size, etc.) after having been at least partially crushed. Crush tolerance is desirable in some examples, for instance, to reduce the risk of prolonged constricting, impinging, or closing of a vessel within which a restrictor apparatus is disposed should pressure or force be applied to the patient in the vicinity of the implanted restrictor apparatus. For instance, for a restrictor apparatus disposed within an ateriovenous graft or fistula, if pressure is applied to the arm (for instance, to reduce bleeding after dialysis), in some examples, it is desirable for the restrictor apparatus to either resist collapsing under the pressure or return to (or substantially return to) a pre-collapsed configuration (shape, size, etc.) after having been at least partially collapsed by the pressure. In this way, the graft or fistula is not partially or totally constricted, impinged, or closed by the restrictor apparatus for a prolonged period of time after the pressure has been removed from the arm of the patient. Such a crush-resistant or crush-tolerant restrictor apparatus can maintain or return to a functioning configuration during or after the applied pressure to reduce the likelihood of having to repair or replace the restrictor apparatus, as well as reduce the chances of the patient sustaining an injury or dying as a result of the restrictor apparatus becoming at least partially crushed or collapsed. In various examples, crush resistance or crush tolerance can be achieved by forming the restrictor apparatus from a self-expanding or shape memory material. In some examples, the restrictor apparatus can be formed from Nitinol. In other examples, it is contemplated that the restrictor apparatus can be formed from other crush-resistant or crush-tolerant materials.

The above Detailed Description includes references to the accompanying drawings, which form a part of the Detailed Description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the term “subject” is used to include the term “patient.” In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more features thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

What is claimed is:

1. A blood flow restrictor apparatus configured to be placed within a destination vessel for an arteriovenous hemodialysis access, the apparatus comprising:
a tubular portion including a delivery configuration and a deployed configuration, the tubular portion being deformably expandable from the delivery configuration to the deployed configuration; and
a size-limiting portion configured to constrain a size of the tubular portion when expanded in the deployed configuration, wherein the tubular portion includes a first portion including a first size substantially matching an interior size of the destination vessel with the tubular portion expanded in the deployed configuration, wherein the tubular portion, when expanded in the deployed configuration, includes a second portion constrained by the size-limiting portion to include a second size smaller than the first size of the tubular portion.

2. The blood flow restrictor apparatus of claim 1, wherein the tubular portion includes a first end and a second end, the first portion of the tubular portion including at least one of the first and second ends of the tubular portion.

3. The blood flow restrictor apparatus of claim 1, wherein the tubular portion includes a stent graft.

4. The blood flow restrictor apparatus of claim 3, wherein the size-limiting portion includes a stent disposed around the tubular portion proximate the second portion.

5. The blood flow restrictor apparatus of claim 3, wherein the size-limiting portion includes a fixed ring disposed around the tubular portion proximate the second portion.

6. The blood flow restrictor apparatus of claim 1, wherein the size-limiting portion includes a stent.

7. The blood flow restrictor apparatus of claim 6, wherein the stent includes a first weave pattern and a second weave pattern, the first weave pattern configured to constrain the first portion of the tubular portion to the first size and the second weave pattern configured to constrain the second portion of the tubular portion to the second size.

8. The blood flow restrictor apparatus of claim 6, wherein the stent includes a first molded portion and a second molded portion, wherein the first molded portion includes a different expansion characteristic from the second molded portion, the first molded portion configured to constrain the first portion of the tubular portion to the first size and the second molded portion configured to constrain the second portion of the tubular portion to the second size.

9. The blood flow restrictor apparatus of claim 1, wherein the first portion of the tubular portion includes the first and second ends, and the second portion of the tubular portion is disposed between the first and second ends.

10. The blood flow restrictor apparatus of claim 1, wherein the destination vessel includes an arteriovenous graft.

11. The blood flow restrictor apparatus of claim 1, wherein the destination vessel includes a vessel of an arteriovenous fistula.

12. The blood flow restrictor apparatus of claim 11, wherein the destination vessel includes a vein of the arteriovenous fistula.

13. The blood flow restrictor apparatus of claim 1, wherein the tubular portion and the size-limiting portion are integrally formed together.

14. The blood flow restrictor apparatus of claim 1, wherein the size-limiting portion is frictionally attached to the tubular portion.

15. The blood flow restrictor apparatus of claim 1, wherein the size-limiting portion is magnetically attached to the tubular portion.

16. The blood flow restrictor apparatus of claim 1, wherein the tubular portion includes a retainer extending from a surface of the tubular portion, the retainer configured to maintain the size-limiting portion in place on the tubular portion.

17. The blood flow restrictor apparatus of claim 16, wherein the tubular portion includes two retainers configured to retain the size-limiting portion in place on the tubular portion between the two retainers.

18. The blood flow restrictor apparatus of claim 1, wherein the tubular portion in the deployed configuration includes:
an entry portion converging from the first size at a first end to the second size of the tubular portion; and
an exit portion diverging from the second size to the first size at a second end of the tubular portion.

19. The blood flow restrictor apparatus of claim 1, comprising an imageable feature configured to be viewable in an imaging modality.

20. The blood flow restrictor apparatus of claim 19, wherein the imageable feature includes a radio opaque feature.

21. The blood flow restrictor apparatus of claim 19, wherein the imageable feature includes an ultrasound reflective feature.

22. The blood flow restrictor apparatus of claim 1, wherein the tubular portion is configured to self-expand from the delivery configuration to the deployed configuration.

23. The blood flow restrictor apparatus of claim 1, wherein the tubular portion is configured to fit over a balloon configured to expand the tubular portion from the delivery configuration to the deployed configuration.

24. The blood flow restrictor apparatus of claim 23, wherein the balloon includes two balloon portions, wherein a first balloon portion is configured to expand the tubular portion proximate a first end, and a second balloon portion is configured to expand the tubular portion proximate a second end.

25. The blood flow restrictor apparatus of claim 1, wherein the blood flow restrictor apparatus, with the tubular portion in the delivery configuration, is configured to fit within a vascular sheath.

26. A method of restricting blood flow for an arteriovenous hemodialysis access, the method comprising:
placing a blood flow restrictor apparatus, in a delivery configuration, within a destination vessel for the arteriovenous hemodialysis access;
expanding the blood flow restrictor apparatus to a deployed configuration, wherein the blood flow restrictor apparatus includes a tubular portion and a size-limiting portion, the size-limiting portion configured to constrain a size of the tubular portion when expanded in the deployed configuration, wherein the tubular portion includes a first portion including a first size substantially matching an interior size of the destination vessel with the tubular portion expanded in the deployed configuration, wherein the tubular portion, when expanded in the deployed configuration, includes a second portion constrained by the size-limiting portion to include a second size smaller than the first size of the tubular portion.

27. The method of claim 26, wherein expanding the blood flow restrictor apparatus to a deployed configuration includes inflating a balloon to expand the blood flow restrictor apparatus.

28. The method of claim 27, wherein inflating the balloon includes inflating two balloon portions, wherein a first balloon portion is configured to expand the tubular portion proximate a first end, and a second balloon portion is configured to expand the tubular portion proximate a second end.

29. The method of claim 26, wherein expanding the blood flow restrictor apparatus to a deployed configuration includes allowing the blood flow restrictor apparatus to self-expand.

30. The method of claim 26, wherein placing the blood flow restrictor apparatus within the destination vessel includes using a catheter to place the blood flow restrictor apparatus within the destination vessel.

31. The method of claim 26, wherein placing the blood flow restrictor apparatus within the destination vessel includes using a vascular sheath to place the blood flow restrictor apparatus within the destination vessel.

32. The method of claim 26, wherein placing the blood flow restrictor apparatus within the destination vessel includes placing the blood flow restrictor apparatus within an arteriovenous graft.

33. The method of claim 26, wherein placing the blood flow restrictor apparatus within the destination vessel includes placing the blood flow restrictor apparatus within a vessel of an arteriovenous fistula.

34. The method of claim 26, wherein placing the blood flow restrictor apparatus within the destination vessel includes placing the blood flow restrictor apparatus within a vein of the arteriovenous fistula.
